(19) United States
(12) Patent Application Publication
(10) Pub. No.: US 2004/0211721 A1
(43) Pub. Date: Oct. 28, 2004

(54) Delivery systems for mycotechnologies, mycofiltration and mycoremediation

(76) Inventor: Paul Edward Stamets, Shelton, WA (US)

Correspondence Address: William R. Hyde, 1833 10th Street, Penrose, CO, 81240, US

(21) Appl. No.: 10/852,948

(22) Filed: May 24, 2004

Related U.S. Application Data
(62) Division of application No. 10/081,562, filed on Feb. 19, 2002.
Application No. 10/081,562 is a continuation-in-part of application No. 09/790,033, filed on Feb. 20, 2001, now abandoned.

(51) Int. Cl. C12N 11/02

(52) U.S. Cl. 210/601; 435/262

(57) ABSTRACT

The present invention utilizes fungal spore mass or hyphal fragments in landscaping cloths, fiber substrates, paper products, hydroseeders and agricultural equipment. The fungi may include saprophytic fungi, including gourmet and medicinal mushrooms, mycorrhizal fungi, entomopathogenic fungi, parasitic fungi and fungi imperfecti. The fungi function as keystone species, delivering benefits to both the microsphere and biosphere. Such fungal delivery systems are useful for purposes including ecological rehabilitation and restoration, preservation and improvement of habitats, filtration of silts and sediments and restoration of abandoned logging roads.

This application is a divisional of U.S. patent application Ser. No. 10/081,562, filed Feb. 19, 2002 (currently co-pending and herein incorporated by reference), which is a continuation-in-part of U.S. patent application Ser. No. 09/790,033, filed Feb. 20, 2001 (now abandoned).

### BACKGROUND OF THE INVENTION

1. Field of the Invention

The present invention is generally related to products and methods for inoculation with beneficial fungi. More particularly, the present invention is related to the use of fungal slurries, landscaping cloths, paper products and mats, hydroseeding equipment and agricultural equipment for inoculation with spores and hyphae of mushrooms and other fungi for purposes including ecological rehabilitation and restoration, bioremediation, habitat preservation and agriculture.

2. Description of the Related Art

The foundation and continuation of life is directly dependent upon healthy habitats. Habitats are increasingly in peril due to the expansion of human enterprises, exacerbating the effects of erosion, and leading to losses in biodiversity and ecological resilience. In the construction of roads, expansion of suburbia and urban centers, trees and shrubs are removed and topsoils are stripped away and soils are compacted. As rains ensue, the forces of erosion further threaten ecological health in removing latent soils and causing sediment accumulation in the lowlands. This severe loss of topsoil tenacity directly results in enormous expenses both societally and environmentally. Certain human enterprises have also resulted in the contamination of widespread areas with toxic wastes and pollutants.

The vegetative, long-lived body of a fungus is an extensive network of microscopic threads (known as mycelium, mycelia or mycelial hyphae) which fully permeates soil, logs, or others substrates within which the organism grows. Most ecologists now recognize that soil health is directly related to the presence, abundance and variety of fungal associations. The mycelial component of topsoil within a typical Douglas fir forest in the Pacific Northwest approaches 10% of the total biomass; the threadlike hyphae of fungal mycelia may exceed one mile of mycelium per cubic inch of soil. Healthy ecosystems include a wide variety of fungal associations. For example, mycorrhizal fungi (including many mushroom fungi) form a mutually dependent, beneficial relationship with the roots of host plants, ranging from trees to grasses to agricultural crops. When the mycelia of these fungi form an exterior sheath covering the roots of the plant they are termed ectomycorrhizal; when they invade the interior root cells of host plants they are called endomycorrhizal (also known as vesicular-arbuscular or VA mycorrhizae). Saprophytic fungi (wood and organic matter decomposers) are the primary decomposers in nature, working in concert with a succession of microorganisms and plants to break down and recycle organic and inorganic compounds and materials. Saprophytic fungi have also been found to form symbiotic, mutually beneficial relationship with a number of agricultural crops. For example, corn is known to give bigger yields in the presence of straw bales inoculated with Stropharia rugosoannulata as compared to uninoculated straw bales. The no-till method of farming also benefits from the growth of Basidiomycetes including mushrooms, reducing plant stubble into nutrients. Parasitic mushrooms have their own role in a healthy ecosystem, although they can become overly destructive in unhealthy systems. Another broad class of decomposers is the more primitive, non-mushroom forming “fungi imperfecti,” including also molds and yeasts.

Evidence of the premier role of fungi as decomposers can easily be gathered in a walk through a healthy forest—rotting logs that have been infested by fungi. Without the presence of fungi, few if any organisms are able to effectively degrade the complex aromatic polymers cellulose and lignin, the two primary components of woody plants; cellulose, and particularly lignin, the most recalcitrant of substrates in nature, are generally otherwise resistant to microbial attack and decomposition. The fungi, particularly “white rot fungi,” which are adept at decomposing lignin, and “brown rot fungi,” premier decomposers of cellulose, produce a complex suite of enzymes that oxidize the structures completely to water and carbon dioxide via a radical-mediated mechanism.

Both liquid substrate and solid substrate cultures of white rot fungi have been the subject of years of bioremediation research in numerous laboratories, as evidenced by the large number of publications and patents in this area. See, for example, U.S. Pat. No. 4,554,075 (1985), U.S. Pat. No. 4,891,320 (1990), U.S. Pat. No. 5,085,998 (1992), U.S. Pat. No. 5,486,474 (1996), U.S. Pat. No. 5,583,041 (1996) and U.S. Pat. No. 5,597,730 (1997). Such saprophytic white rot wood-decomposing fungi have shown the ability to degrade recalcitrant foreign compounds such as polynuclear aromatic hydrocarbons (PAHs), alkanes, creosote, pentachlorophenol (PCP), polychlorinated biphenyls (PCBs), dichlorodiphenyltrichloroethane (DDT), trinitrotoluene (TNT), dioxin, nitrogenous compounds such as ammonium nitrate, urea, purines and putriscines, as well as agricultural wastes and agricultural runoff. However, these bioremediation processes have significant limitations, hindering the transition from the laboratory to large scale field applications, and in general have not been used commercially. One particular problem has been that economic and effective delivery systems for large scale field applications of white rot fungi have not been available.

The saprophytic fungi have also proven to be efficient digesters of potentially harmful organisms such as coliform bacteria and nematodes. The voracious Oyster mushrooms (Pleurotus ostreatus) have been found to be parasitic against nematodes. Extracellular enzymes act like an anesthetic and stun the nematodes, thus allowing the invasion of the mycelium directly into their immobilized bodies.

For these and other reasons there has been great interest in fungi for uses such as introduction of mycorrhizal fungi, bioaugmentation of soils, bioremediation, biological control and production of mushrooms.

Among the methods for delivering fungal spores and hyphal inoculum to soil for various purposes such as bioremediation or agriculture are carriers such as grain, sawdust and wood chip spawn, alginate hydrogels with and without additional nutrient sources, vermiculite and peat optionally saturated with nutrient broths, vermiculite and rice flour or grain flour, straw or other agricultural waste products overgrown with fungal mycelium, pelleted fungal inoculum preparations, etc.

The usual methods for inoculation with fungi are typically expensive, labor intensive and/or ineffective. Various techniques have been used to inoculate growing substrates with those fungi known as mushrooms. These include methods of inoculating beds of wood chips, beds of compost, lawns and soils. Also known are methods of inoculating soils with fungi for the bioremediation purposes.

Beds of wood chips are typically inoculated by spreading sawdust and/or woodchip spawn (spawn being defined herein as any material inoculated with mycelium or impregnated with mycelium and used for inoculation) throughout the wood chips or by placing a layer of spawn within the wood chips. Beds of compost are typically inoculated in a similar manner with a grain spawn, although a sawdust spawn may also be utilized in some instances. The use of expensive spawn of limited shelf life produced by labor- and equipment-intensive sterile culture methods are among the disadvantages of this approach.

Another method of inoculation involves spore mass inoculation or inoculation with mycelia fermented under sterile conditions. In the first method spores may be collected and broadcast, but more preferably is conducted by immersion of the mushroom(s) in water to create a spore mass slurry, the addition of molasses, sugars and/or sawdust to stimulate spore germination, aeration, incubation and broadcast of the aqueous spore mass slurries. This approach and the similar approach with liquid mycelium inoculated and grown under sterile conditions may be successfully utilized. These approaches, however, require either fresh spore-producing mushrooms or sterile culture techniques, and application must be during the time frame of vigorous peak growth after germination or inoculation or the mycelial fragments will not coalesce into a contiguous mycelial mat. There remains a need for more convenient products and processes for widespread application of biologically active spore and/or mycelial inocula.

Trees, lawns and seedbeds have been inoculated with mycorrhizal species using various tablets or gels prepared from spores or mycelium. Trees may also be inoculated with mycorrhizal mushrooms by dusting the roots of seedlings with spores or mushroom mycelium or by dipping the exposed roots of seedlings into water enriched with the spore mass of the mycorrhizal species. Another method for inoculating mycorrhizae calls for the planting of young seedlings near the root zones of proven mushroom-producing trees, allowing the seedlings to become ‘infected’ with the mycorrhizae of a neighboring tree. After a few years, the new trees are dug up and transplanted. Another method involves broadcasting spore mass onto the root zones of trees. Such approaches can be labor intensive, expensive, of uncertain success and/or not suited to widespread use.

Patented approaches for inoculation with mycorrhizal fungi include U.S. Pat. No. 4,294,037 (1981) to Mosse et al. for a process for the production of vesicular-arbuscular (VA) mycorrhizal fungi comprising growing a VA fungus on plant roots in nutrient film culture for 1 to 3 months and harvesting for inoculum production; U.S. Pat. No. 5,178,642 (1993) to Janerette for culturing of ectomycorrhizal fungal inoculants on a solid medium, contacting the mycelia in the solid medium with perlite wetted with a nutrient solution, incubating for about three months and broadcasting; and U.S. Pat. No. 4,551,165 (1985) to Warner for mycorrhizal seed pellets formed from vesicular-arbuscular mycorrhizal inoculum peat, at least one seed and a binder compacted into pellet form. It is also known to add various compositions to seeds to assist growth. For example, U.S. Pat. No. 5,586,411 (1996) to Gleddie et al. discloses methods for adding Penicillium bilaii and Rhizobium bacteria in a sterilized peat base to legume seeds so as to increase the availability of soluble phosphate and fixed nitrogen. However, it is not known to add mycorrhizal fungi directly to seeds, nor is it known to combine saprophytic or entomopathogenic fungi directly with seeds or seedlings, nor is it known to combine mycorrhizal fungi with saprophytic, entomopathogenic and/or imperfect fungi for the purpose of habitat restoration. Again, there remains a need for cheaper and more efficacious methods for large scale use.

U.S. Pat. No. 6,033,559 discloses microbial mats constructed of stratified layers of cyanobacteria and purple autotrophic bacteria, and optionally other microorganisms such as algae or fungi, organized into a layered structure held together with slime with an organic nutrient source provided, optionally with support structures such as shredded coconut hulls, ground corn cobs or wood fiber. While such bacterial mats may be suited to aquatic environments, they are not particularly suited for terrestrial applications. An additional disadvantage is that algae are generally not as ‘enzymatically equipped’ to deal with toxins and pollutants, the fungi being the keystone species which render nutrients available to the photosynthetic, chlorophyll producing algae and plants.

Trends in spawn technology have long been evolving towards pelletized or granular spawn, for purposes such as inoculation of substrates for production of gourmet and medicinal mushrooms, inoculation with mycorrhizal fungi, inoculation with white rot fungi for bioremediation and inoculation with fungi imperfecti for control of soilborne pathogens. Various forms of pelletized spawn are known, including those formed from nutrients, with or without binders, and peat moss, vermiculite, alginate gel, alginate gel with wheat bran and calcium salts, hydrophilic materials such as hydrogel, perlite, diatomaceous earth, mineral wool, clay, etc. See Stamets, Growing Gourmet and Medicinal Mushrooms (1993) and U.S. Pat. No. 4,551,165 (1985), U.S. Pat. No. 4,668,512 (1987), U.S. Pat. No. 4,724,147 (1988), U.S. Pat. No. 4,818,530 (1989), U.S. Pat. No. 5,068,105 (1991), U.S. Pat. No. 5,786,188 (1998) and U.S. Pat. No. 6,143,549 (2000). Pelletized spawn is specifically designed to accelerate the colonization process subsequent to inoculation. Examples of pelletized spawn range from a form resembling rabbit food to pumice-like particles.

Idealized pelletized spawn seeks a balance between surface area, nutritional content, and gas exchange and enables easy dispersal of mycelium throughout the substrate, quick recovery from the concussion of inoculation, and sustained growth of mycelium sufficient to fully colonize the substrate. Many grains and other substrates are, however, pound-for-pound, particle for particle, more nutritious than most forms of pelletized spawn. Furthermore, use of grains or liquid-inoculum or other forms of inoculum avoids the expense and labor of pelletizing. There remains a need for more economical and more efficacious means of inoculation of large scale areas.

It is known that berms and revetments and other protective structures are employed to halt soil erosion caused by runoff or precipitation. One particular, well-known system for the creation of such protective structures consists in the construction and use of “gabions,” e.g., “mattress gabions,” large, thin rectangular containers filled with gravel, crushed stone and other material, fitted with a cover and consisting of galvanized or galvanized and plastic-coated wire netting panels joined together with ties or wire stitches and designed to cover, without any break, extensive tracts of land of the most disparate conformation, as if they were actual ‘mattresses.’ Similar structures may be constructed of “basket gabions,” “sack gabions,” “gabion mats” and “log gabions.”

In many applications, there is a need for gabions to rehabilitate the environment and allow development of an ecosystem able to utilize the water runoff, thereby resisting erosion in a more environmentally sound manner. In other applications, a gabion that is biodegradable would be more useful than those metal or other degradation-resistant materials used to construct gabions. There is also a need for gabions that could ‘filter’ contaminants such as agricultural runoff, including fertilizer, animal waste and pesticide runoff, urban runoff, etc. for protection of streams and rivers. In many situations there is also a need for gabions of cheaper materials.

There is, therefore, a continuing need for enhancing the effectiveness of fungal inoculation and growth and thereby improving habitat preservation and habitat recovery. There is also a need for enhanced products and methods for accomplishing fungal inoculation as an aid to such and habitat recovery and preservation. There is also a need for such fungal products and methods as an aid to agriculture, including both plant cultivation and mushroom cultivation.

In view of the foregoing disadvantages inherent in the known types of fungal inoculants, the present invention provides improved inoculating agents and methods of using such agents.

### BRIEF SUMMARY OF THE INVENTION

Fungi have been found by the present inventor to be a “keystone species,” one that facilitates a cascade of other biological processes that contribute to healthy ecologies, the fungi being necessary for health of environments and capable of “leading the way” in the remediation, reclamation, restoration and/or preservation of environments. As fungi, including many or all gourmet and medicinal saprophytic mushroom fungi, produce extracellular enzymes and acids not only capable of breaking down cellulose and lignin, but also hydrocarbons such as oils, petroleum products, fuels, propellants, PCBs and many other pollutants, the fungi are particularly suited to bioremediation of badly polluted and eroded environments, depleted environments, etc. Such fungi have also been found to be a keystone in the most healthy and luxuriant terrestrial environments. Fungal organisms are now known as the largest biological entities on the planet, with various individual mats covering more than 20,000 acres, weighing 10,000 kg. (22,000 lb.) and remaining genetically stable for more than 1,500 years. The momentum of mycelial mass from a single mushroom species, growing outwards at one-quarter to two inches per day, staggers the imagination. These silent mycelial tsunamis affect all biological systems upon which they are dependent. As one fungus matures and dies back, a panoply of other fungi come into play, acting to catalyze habitat recovery and habitat health.

Nearly all plants have joined with saprophytic and mycorrhizal fungi in symbiosis. Plants may devote a majority of the net energy fixed as sunlight to below ground processes, not only root growth but also to feed mycorrhizal fungi and other microorganisms. However, this symbiotic relationship is not a net energy loss. Mycorrhizal fungi surround and penetrate the roots of grasses, shrubs, trees, crops and other plants, expanding the absorption zone ten- to a hundred-fold, aiding in plants' quest for water, transferring and cycling macro and micro nutrients, increasing soil aeration and the moisture-holding capacity of soils and forestalling blights, pathogens and disease. With the loss of fungi, the diversity of insects, birds, flowering plants and mammals begins to suffer, humidity drops, now-exposed soils are blown away, and deserts encroach. To aid in the solution of these problems, new “mycotechnologies” (after mycology, the study of fungi) are provided herein.

In view of the disadvantages inherent in the known products and methods for fungal inoculation, the present invention provides improved products and methods for intensive and/or widespread inoculation of beneficial fungal species. The present invention provides new products and methods utilizing fungal spore and hyphal compositions, useful for impregnation of soils, fabric landscaping cloths, soil blankets and rugs, mats, mattings, bags, gabions, fiber logs, fiber ropes, fiber bricks, etc.; useful for distribution via spray hydroseeding equipment and mobile hydroseeders; useful for agricultural planting equipment, harvesting equipment and field preparation equipment; useful for cultivation of gourmet and medicinal mushrooms; and useful for the habitat restoration and preservation uses described herein. Inoculation with beneficial fungal spores and/or mycelial hyphae, and optionally and preferably with seeds, provides products and methods useful for purposes including enhancing plant growth and mycorrhizal and symbiotic relationships, habitat restoration, erosion control and stabilization of soils, treatment of contaminated habitats, filtration (“mycofiltration”) of agricultural and urban water runoff, fungal bioremediation (“mycoremediation”) of biological and chemical pollutants and toxic wastes, and production of mycelia and mushrooms and improved production of plants, providing nutrients to insects, herbivores and numerous organisms up and down the food chain. Preferred fungi include the “fungi perfecti” (including those fungi producing gilled and polypore and other mushrooms) and the “fungi imperfecti” (the simpler, non-mushroom producing fungi including molds and yeasts) and their various forms of mycelium and spores, including both sexually produced and asexually produced spores and spore variations. Particularly useful are the saprophytic mushrooms for purposes such as mycoremediation and mycofiltration of agricultural and urban runoff, the saprophytic and mycorrhizal fungi for improvements in agricultural products and methods, the entomopathogenic fungi for insect control, and combinations of the saprophytic, mycorrhizal, entomopathogenic and/or other fungi imperfecti. Such products and methods further provide reduced costs, ease of application and improved efficiency when compared to known products and processes.

The fungal inoculation products and the fungal methods of the present invention may, depending upon the application, advantageously include habitat recovery and restoration, erosion control, rapid decay and decomposition of forest debris and agricultural waste, bioremediation of contaminated sites through decomposition of hydrocarbon based contaminants and concentration/removal of heavy metals from soils, adjustment of soil pH, mycofiltration of agricultural and industrial runoff, large-scale introduction of mycorrhizal species, gourmet species and other beneficial mushroom species, introduction of entomopathogenic (capable of causing disease in insects) fungi for control of pest insects, fungi for control of soilborne plant pathogens, the production of gourmet and medicinal mushrooms, and numerous other applications. A water-spore, water-mycelial hyphae or water-spore and/or hyphae-seed slurry (or similar slurries with vegetable or other oils) may be applied directly to soils. Alternatively, the water-spore, water-mycelial hyphae or water-spore-hyphae or oils suspension is applied to commercially available products such as landscaping cloths, gabions, mats, burlap and other fiber bags, paper and/or cardboard materials, bulk substrates or other fiber substrates, etc., optionally simultaneously with or followed by seed application. As another alternative, such products may be inoculated by traditional inoculation methods, such as those utilizing grain spawn or sawdust spawn. Less preferably, similar products made of non-biodegradable materials may be utilized. A water-seed-spore mass or water-seed-mycelial hyphae slurry offers a novel approach for inoculating environments with fungi and can be applied directly to bare soils, straw, reeds, wood chips, sawdust, fibers and fiber products, landscape fabrics and papers, burlap sacks, gabions, etc. The mycelial hyphae may be utilized fresh, dried or freeze-dried. The benefits of these products and approaches include ease of application, erosion control, habitat restoration, mycofiltration, mycoremediation, and mycorrhizal and fungal associations.

The use of such aforementioned fungally impregnated biodegradable membranes, in combination with plant seeds allows for a unique delivery system: cardboard boxes whose side walls have been infused or applied with plant seeds in combination with fungal spores, mycelium, or extracts of the mycelium of mycorrhizal, symbiotic, saprophytic, and entomopathogenic fungi. A multiplicity of problems are solved with one solution. The prevalence of cardboard boxes delivered throughout the world on a daily basis exceeds thousands of tons per day, boggling the imagination. The cardboard box is ubiquitous to the world community. The predominance of cardboard in the manufacturing of boxes and its over-abundance strains the resources of communities. With this invention, cardboard boxes have a value-added, after market benefit as they become a living resource for ecological recovery. The panels of the box can be used for home gardening, commercial agriculture, for mycofiltration, mycoremediation, and mycopesticidal purposes. The box can be used as an educational tool for teaching children while at the same time be the container for transporting items related or unrelated to the invention. The cardboard boxes become an ecological footprint for creating a garden, seed bed, an orchard, a forest and even an expanding oasis, starting the process of habitat improvement and recovery. An added advantage is that the cardboard panels can be placed over soil to suppress competitive weed growth and to retain moisture. The decomposition of the paper based materials by the fungus releases nutrients to aid plant growth.

Oils may also be used as a carrier material. Petroleum oils can be readily digested by certain fungi and biodegradable oils are readily digested by most or all fungi perfecti and fungi imperfecti. Therefore oil-spore or oil-hyphae mixtures or water-oil-spore or water-oil-hyphae suspensions, with or without seeds, provide an alternative to the water-spore or water-hyphae slurries which may be utilized in the practice of the present invention. In general, biodegradable oils are preferred as offering an environmentally friendly and a more readily available nutritional source to a wide variety of fungi. Such fungal or hyphal oils may also be preferably employed in applications such as ecological rehabilitation, mycoremediation and mushroom growing where use of a vegetable oil as an additional nutritional source is desired.

The use of fungi as keystone organisms releases nutrients into the surrounding environment from the biodegradable carrier materials to enhance the growth of targeted or naturally occurring plants, from grasses to shrubs to trees to complex biological communities. In essence, biological successionism can be directed through the use of a single species or a complex plurality of fungal components, using fungi as the keystone organisms leading the way in habitat enhancement or recovery. The fungi may optionally be used in combination with plants, algae, lichen, bacteria, etc.

Biodegradable fabric cloths and blankets made of straw, coconut fibers, corn stalks, wood fibers and other similar materials, wood chips and straw bales are in common use along roadsides to help prevent or lessen erosion and help ecological recovery. When plant root growth increases in these locations, the tenacity of the soil is enhanced, lessening the chances for erosion. However, none use a fungal component as a determining factor in enhancing the effects of such biodegradable erosion-control materials. The present invention offers improved products wherein fungi act as a “keystone” or “linchpin” species, ameliorating the impact of erosive forces by helping to establish communities of organisms, using fungi to enhance or control the growth of other organisms including but not limited to plants, protozoa, bacteria, viruses, algae, lichens, invertebrates, arthropods, worms and/or insects. Also advantageous is the use of fungal mycelium to enhance the tenacity of overlaying fabric cloths or bulk substrate on habitats, thus preventing ‘slippage’ and anchoring the fabric cloths, wood chips, straw, etc.

Such mycelial products are also useful for combating viruses and virulent bacteria, for example Escheria coli, Bacillus subtilis, malaria, cholera, anthrax, and water-borne diseases, as well as biological warfare (BW) pathogenic species. By infusing mycelium into cloths, blankets, gabions, mats, berms, etc., targeted disease organisms such as bacteria, fungi, viruses, protozoa and amoebas can be effectively reduced, ameliorating the downstream impact as well as in residence. Such benefits could help fisheries, for instance, stave off Pfiesteria.

In another embodiment of the present invention, fungal spores and/or mycelial hyphae are introduced into hydroseeding equipment, agricultural seeding equipment, harvesting equipment and other agricultural equipment. This allows for the simultaneous inoculation of beneficial fungi directly into lawns, disturbed soils, agricultural fields, agricultural wastes, etc.

The addition of fungal tissue (spore mass and/or hyphae) into landscaping materials, hydroseeding-type equipment and all types of agricultural equipment is an effective means for the simultaneous replanting and fungal inoculation of disturbed or recovering environments, leading to habitat restoration, improved control of runoff and mycofiltration of runoff (trapping biological and chemical contaminants, denaturing them), etc. The addition of fungal inocula to agricultural equipment can provide improved means of introducing beneficial symbiotic saprophytic fungi and mycorrhizal fungi, entomopathogenic fungi for control of insect pests and fungi imperfecti for control of soilborne plant pathogens. Introduction of such fungal inocula into harvesting equipment can provide efficient means of inoculating agricultural waste products or efficient production of inoculated straw bales and rounds, etc., useful for the practice of many embodiments of the present inventions.

Another advantage of the present invention lies in the use of fungal components to accelerate the decomposition of biodegradable fabrics and other materials in sensitive environments where such fabrics and materials have been placed for the purposes of preventing erosion and enhancing habitat recovery.

Another advantage of the present invention arises from the use of fungal components in biodegradable materials to enhance water retention properties of such materials, using the natural water-absorption properties of mycelium.

Supplementary advantages arise from the fact that fungally colonized mycelial fiber substrates liberate carbon dioxide, essential for healthy plant growth, especially essential for young seedlings. As the grass or other plants grow up, it creates a high humidity layer through condensation formation from dew point as well as the ‘greening’ effect which is naturally cooler.

Further advantages arise from the use of adsorbent or absorbent biodegradable fiber cloths and mats inoculated with spores and/or hyphae of petroleum oil-eating fungi. Thus the oil slicks or spills may be soaked up by the cloth or mat material and digested by the mycelium of the fungus.

An additional advantage is the use of fungally impregnated biodegradable materials along stream and sensitive watersheds to ameliorate the impact of runoff containing sediment and pollutants. The use of such products allows for sequestration of excess or harmful nitrogenous, phosphorus-laden or carbonaceous compounds as well as sediment and silt from gravel roads and other sources. Fisheries, especially spawning streams of salmon and trout, as well as other species such as shellfish, benefit directly and dramatically from mycofiltration of silt and sediment, which can create an environment inhospitable to eggs, and pollutants, which can have far-ranging negative effects. Numerous advantages naturally follow the use of such mycelial products and methods to protect sensitive watersheds such as salmon and trout spawning grounds, riparian runoff and wetlands, thereby providing mushroom and mycelial biomass which then feeds developing larvae of numerous insects, providing additional benefit to fisheries and recreational users through enhancement of the food chain as well as through protection from upland runoff.

The present invention provides further advantages via use of a fungal component or components in biodegradable materials to help catalyze significant climate change in arid environments through the enhancement of the water retention capacities of the top soils, leading to the ‘oasis’ phenomena in dryland habitats, the net effects of which are not only erosion control, but significant enhancement of biological communities which then can become ‘seed’ banks leading to a creations of satellite communities in proximity to the genome source.

Another advantage of the present invention is the use of fungal components in biodegradable materials to create communities of fungi, including commercially valuable mushrooms.

Additional advantages arise when such products and methods are used to bioremediate contaminated, toxic and hazardous sites, providing breakdown of dangerous organic, inorganic and biological threats while simultaneously triggering the ecosystem recovery as above. In biologically hostile environments, a small sample of the targeted habitat can be introduced to the fermentation of the fungal mycelia, at a late stage, so that the chosen fungal candidate can acclimate to the complex biota of the targeted environment. This technique reduces transplant shock, and further enhances the effectiveness of the present invention.

Further advantages arise from the use of colonized fiber substrates to combat virulent bacteria, reduce or eliminate viruses, limit pathogenic fungi, yeasts, and molds, control protozoa such as amoebas, ciliates, flagellates, and sporozoans, control multicellular organisms such as rotifers and trap and digest nematodes.

Further advantages are obtained when such ‘mycocloths’ and ‘mycomats’ are infused with fungi capable of decomposing biological and chemical warfare toxins. The mycocloths and mycomats can then be used to decontaminate toxic landscapes, battlefield and otherwise, thus leading to reuse of valuable land.

Still further advantage may be gained from use of fungally impregnated biodegradable materials, either contained within or in the absence of a matrix of biodegradable or non-biodegradable materials, to concentrate heavy metals, for example radioactive metals and precious metals, which then can be removed to eliminate toxins topically and subsurface. Such residual organic debris and mycelia could be economically or profitably separated from the metals through incineration, biodigestion with other organisms (e.g., bacteria, protozoa or yeasts) and or via chemical treatments (e.g., enzymes, acids or catalysts).

The present invention provides further advantages through use of entomopathogenic fungal components to control, reduce or eliminate pest insects or disease-carrying insects in the applied environments. Extracts of the pre-conidial mycelium of entomopathogenic fungi may also be utilized to attract and/or control insects. More broadly, fungal components in biodegradable materials may be utilized to control harmful insects, enhance insect communities, or invite beneficial insects in the applied environments. Since insect communities can influence or predetermine bird communities, the fungal constituent has a direct downstream effect on this and many other biological successions.

The present invention thus allows for wide scale inoculation of desired mushroom species on widely varying substrates suitable for use in various applications and environments. Numerous advantages arise from growing beneficial fungi and mushrooms for various agricultural, forestry, ecological and bioremediation purposes including habitat restoration and preservation, rapid decay of forestry byproducts and wastes, mycofiltration of agricultural and industrial runoff, decomposition of hydrocarbon based contaminants and toxins, concentration/removal of heavy metals from soils, sewage or other substrates, insect, pest and disease control, soil improvement and adjustment of soil pH, introduction of mycorrhizal fungi, production of gourmet and medicinal mushrooms, improved crop yields, etc.

The present invention has been found to achieve these advantages. Still further objects and advantages of this invention will become more apparent from the following detailed description and appended claims. Before explaining the disclosed embodiments of the present invention in detail, it is to be understood that the invention is not limited in its application to the details of the particular products and methods illustrated, since the invention is capable of other embodiments which will be readily apparent to those skilled in the art. Also, the terminology used herein is for the purpose of description and not of limitation.

### DETAILED DESCRIPTION OF THE INVENTION

Innovations of the present invention include introducing saprophytic fungi, mycorrhizal fungi, entomopathogenic fungi, fungi imperfecti and/or other fungi as keystone species using a wide variety of novel products and methods. By infusing substrates or soils with fungal inoculum as disclosed herein, widespread areas of land, sensitive areas such as stream banks and riparian areas, drainages into wetlands, areas in need of topsoil supplementation, polluted areas, etc. may be favorably treated and transformed via fungi. By selecting the type of fungal spores or hyphae to be infused, an ecologist, remediator, forester, farmer, landscaper and others can direct the course of ecological recovery or ecological preservation, thereby improving the economical usefulness of the land for varying forest, farm, riparian, agricultural and urban uses. Furthermore, by selecting the types of seeds, persons can further direct the course of development—for example, by using a mixture of grasses and trees, the grasses typically germinating first followed by germination of the tree seeds. Alternatively, seedlings may be directly utilized. Such fungal inoculation may be accomplished via fibrous fabrics, hydroseeding equipment or a variety of agricultural equipment.

In one embodiment, spores, spore mass, actively growing mycelial hyphae, dried or freeze dried powdered fungal mycelium, and/or powdered mushroom fruitbodies are placed into carrier materials used for landscaping and ecological purposes. Mycorrhizal fungi and/or various wood, lawn and field mushrooms and/or entomopathogenic fungi and/or fungi imperfecti may be utilized. The landscaping carrier materials are preferably also impregnated with the seeds of grasses, native grasses, flowers, native wildflowers, and/or trees and other plants. Although some seeds may become ‘fungi food,’ particularly when fresh live mycelium is utilized, some seeds will survive and germinate. Alternatively, such landscaping carrier products may be inoculated, overgrown with mycelium, and seeds then added. Additional organisms such as bacteria, lichens, moss, algae, etc., as well as other fungi, both perfect and imperfect, may optionally be added. Such mats or larger fabrics or other fiber products may be overlaid onto disturbed grounds both to aid plant growth and as a vehicle for treating contaminated habitats, wherein the mycelium acts as a mycofiltration membrane, trapping biological and chemical contaminants and denaturing them. Similarly, a wide variety of landscaping carrier products, discussed in more detail below, may similarly be utilized. The present invention also includes kits for the construction of such fabrics, mats and other fiber carrier products.

Mycomaterials which are utilized after being overgrown with mycelium may be utilized fresh or metabolically arrested via refrigeration for storage and transport. Alternatively, the mycelium may be metabolically arrested through freeze-drying (flash chilling), drying, or by other means, for storage, transportation and subsequent rehydration for field deployment. Storage time of up to a year or more is possible. It will be understood that such metabolic arresting of development may encompass either a slowing of metabolism and development (such as refrigeration) or a total suspension or shutdown of metabolism (freeze-drying, air-drying and cryogenic suspension).

The novel fungal inoculum/ seed sprays and slurries may be applied directly to soils. For many applications it is preferable to apply fungal inoculum to landscaping materials such as wood or straw bulk substrates, mulches, biodegradable landscaping fabrics and blankets, mats, bags, gabions, fiber baskets, fiber-logs, fiber-bricks, cardboard, paper, etc., thereby providing an initial nutritional source, particularly in applications such as habitat restoration, erosion control, mycoremediation, mycofiltration, landscaping, etc.

The mycotechnologies of the present invention may be utilized in the various states of fungal lifecycle, with or without seeds. Where a landscaping type application is desired, a preferred embodiment will often be a paper, cardboard or fabric cloth-seed-spore and/or mycelial hyphae embodiment, with germination of spores, hyphae and seeds occurring upon placement and watering or rainfall. Such may also be preferred in certain erosion control and habitat preservation or rehabilitation applications. For other applications, such as mycoremediation, berm building and mushroom cultivation, mycocloths overgrown with live fungal mycelium on thicker, more rug-like or mat-like materials may sometimes be preferred. For these and other applications, it may be preferable to form a fibrous material, such as burlap, into a sack or bag, or to form a thicker material into bags, basket gabions or mattress gabions and fill with woody fiber and/or non-woody fiber materials. Such sacks, bags and gabions, and optionally their contents, may be inoculated with spores, fresh mycelial hyphal fragments, dried or freeze-dried mycelial hyphae, powdered mushrooms or spawn or combinations thereof, and utilized either immediately after inoculation or after the fibrous material has been overgrown by hyphae, depending on circumstances and desired use. The mats may be deployed in various settings, including both terrestrial and aquatic (such as floating mats). Mycomaterials which are not initially combined with seeds may later have seeds or growing plants added, for combined efficacy with the fungal component for bioremediation, erosion control, landscaping aesthetics, etc.

Suitable landscaping and/or non-landscaping materials, carriers and spawn products include geocloths and geofabrics, soil blankets, landscaping fabrics and other fabrics, nettings, rugs, mats, mattings, fiber felt pads, straw tatamis, mattress inserts, burlap bags, papers, fiber logs, fiber bricks, gabions, cardboards, papers, etc. These materials, carriers and products may be formulated of any suitable fiber, including those derived from woody and non-woody fibers such as wood chips, sawdust, wood pulp, wood mulch, wood wastes, leaf paper, wood-based papers, non-wood papers, pressed cardboard, corrugated cardboard, fiberized rag stock, cellophane, hemp and hemp-like materials, bamboo, papyrus, jute, flax, sisal, coconut fibers, wheat straw, rice straw, rye straw, oat straw and other cereal straws, reeds, rye grass and other grasses, grain hulls and other seed hulls such as cottonseed hulls, cornstalks, corncobs, soybean roughage, coffee plant waste and pulp, sugar cane bagasse, banana fronds, palm leaves, the hulls of nuts such as almonds, walnuts, sunflower, pecans, peanuts, etc., soy waste, cactus waste, tea leaves and the wide variety of other agricultural waste products and combinations thereof. Suitable animal fibers include wool, hair and hide (leather) and combinations thereof. In general, biodegradable wood or plant fibers are preferred over non-biodegradable synthetic fibers. Such is particularly the case with fabrics, mats, blankets, bags, gabions, fiber-logs, etc. utilized for purposes such as mycoremediation, mycofiltration, construction of biodegradable berms, levees, revetments, embankments, etc. Suitable synthetic fibers include plastics and polymers such as polypropylene, polyethylene, nylon, etc. The fibrous woody and non-woody plant fibers may be in any form including paper, textile, fabric, veil, mat, matted, mesh matting, matting rug, felt pressing, blanket, filter, woven, woven roving, open weave, nonwoven, knitted, strand roving, continuous strand, chopped strand, knotted, yarn, braided ropes, milled fiber, high-pressure extrusion rope or mat, composites, etc. and combinations thereof.

Carrier materials may optionally be amended to provide additional nutrients via spraying or soaking of the materials in sugars such as maltose, glucose, fructose or sucrose, molasses, sorghum, mannitol, sorbitol, corn steep liquor, corn meal and soybean meal, vegetable oils, casein hydrolysate, grain brans, grape pumice, ammonium salts, amino acids, yeast extract, vitamins, etc. and combinations thereof. Typically such amendments should be utilized sparingly or with materials that are to be pasteurized or sterilized, as such amendments, particularly carbohydrates and nitrogen supplements, may greatly reduce substrate semi-selectivity for fungi and increase the risk of contamination after fungal inoculation.

Carrier materials such as cardboard panels or other paper-based membranes, can be inoculated with fungi and plant seeds. Such panels can be incorporated into the manufacturing of boxes, especially cardboard boxes. If mycorrhizal, saprophytic and/or mycopesticidal fungi are used in concert with compatible seeds of plants, the cardboard panels become springboards for life and ecological recovery. Fibers selecting from the group consisting of paper pulp fibers, cellophanes (including those with silicon fibers), shredded paper products, wood fibers, sawdusts, corn, jute, coir, coconut, hemp, wheat, rice, grasses, coffee, cotton, kenaf, mosses, lichens, mugworts, wools, animal skins, and biodegradable polymers can also be utilized for the construction of membranes or box panels incorporating this invention. The aforementioned materials can be reformulated to incorporate fungi in the form of spores or mycelium in combination with plant seeds. The boxes still serve their traditional, structural function for the delivery of goods, but now have increased value for their after-delivery use. The panels or boxes could be used for other purposes unrelated to this invention, and increased value because of its further utility in growing plants, enhancing food production and for bioremediation. The panels of the box host assortments of seeds customized to the ecological and cultural specifics of their destination. The selection of seeds predetermines the selection of mycorrhizal and saprophytic fungi. Upon unpacking the box's contents, the box is disassembled by hand or by sharp instrument. The cardboard panels, infused with seeds and fungi, are laid upon or into soil. With the addition of water, the cardboard softens, the fungi are activated, and the seeds germinate. Immediately upon germination the seeds have contact with beneficial fungi, insuring an early symbiotic relationship before competitor fungi can harm the seeds. The mycorrhizal fungi stimulate shoot and root growth, expand the sphere of the root zone for absorption of water and nutrients, improve the micro-hydrology of the surrounding soil, and protect the young plants from diseases. With moisture, the saprophytic fungi decompose the cardboard, freeing more nutrients. The cardboard layer lessens evaporation, preserves moisture, shades and cools the soil underneath. The softening cardboard allows the penetration of the shoots and roots. If the cardboard is scored with fine cuts during manufacturing, the roots and shoots can emerge unencumbered. The cardboard fully decomposes, becoming soil, and leaves no waste.

One of the many useful applications of this ‘living box’, that is, a box constructed with dormant fungi and seeds, for assisting refugees, indigenous displaced peoples, including victims from natural and man-made disasters. As the first emergency relief often is delivered to refugees in a box, there is the economically feasible opportunity of utilizing the delivery box as inoculum for growing plants and fungi. The insides of the box could be sorted according to species of plants, climatic zones, pH requirements, and soil conditions. By example but not by limitation, the seeds of the plant species could be selected from the group comprising of corn, wheat, rice, oats, rye, lentils, beans, squash, melons, potatoes, carrots, turnips, garlic, ginger, mustard, chard, cilantro, fennel, oregano, chives, basil, thyme, and onions. Such box panels would be recognized by the recipients as having a value, a natural currency for anyone who has an interest in cultivating and habitat recovery. The educational lesson from having children using the ‘living box’ is as important an advantage of this invention as any aspect previously described.

The use of cloths, rugs, mats, papers, cardboards, etc. for fungal inoculation products and methods makes advantageous use of several fungal characteristics. For example, it has been found by the present inventor that quite different techniques are called for when inoculating soils and non-sterile substrates as compared to sterile substrates. When inoculating sterilized or pasteurized substrates, or materials composted so as to prepare a selective nutritious medium of such characteristics that the growth of mushroom mycelium is promoted to the practical exclusion of competitor organisms (see The Mushroom Cultivator (1983) by Stamets and Chilton), a technique known as “through spawning” is preferable, wherein the fungal inoculum is introduced via numerous inoculation points (such as colonized grain spawn or sawdust spawn) throughout the medium. However, such an approach in non-sterile bulk substrates such as wood chips or soil may lead to disaster. Each inoculation point becomes a separate colony surrounded by competitor organisms in all directions, often with the result that the inoculation points are unable to generate the necessary mycelial momentum to successfully colonize the substrate. The present inventor has found “layer spawning” or “sheet inoculation,” wherein the fungal inoculum is spread in a horizontal layer within the non-sterile bulk substrate, to be much more successful. Such sheet inoculation takes advantage of several fungal characteristics: 1) mycelia often grows and spreads most rapidly in the lateral, horizontal directions; 2) when mycelia grows horizontally and links into a mycelial layer or mat, it becomes much more vigorous, resistant to contaminants and competitive, allowing further successful growth and colonization in the vertical direction; and 3) ‘wild’ mycelial organisms are typically matlike and layered in that they may cover many acres, yet be only a few inches deep. Thus a landscaping cloth or mat introduces inoculation points and allows for horizontal growth in accord with the mushroom or fungi's natural characteristics. By having a contiguous sheet of mycelium above toxins, extracellular enzymes can “rain” down, effectively decomposing them.

It has further been found that when “sandwich inoculation” utilizing two or more such layers of inoculum is utilized, competitiveness and ultimate success is even further enhanced as the two mycelial layers grow vertically and link up, forming a thoroughly colonized block. In such cases, having two (or more) layers of fungal inoculum with substrates sandwiched in between gives more resilience, allowing for more duration, increasing effectiveness over the long term. Thus when mycelial landscaping cloths or mycelial mats are preferred, a plurality of mats or cloths in stacked, separated layers will often be even more preferable. It will be noted that when cloths are formed into a bag or sack, inoculated with spores or hyphae, and filled with bulk substrates such as woodchips, two lateral layers of cloth are naturally formed, plus a route for initial vertical growth and linkup of layers is provided. Thus in many application, such ‘mycobags’ will be preferred. Such mycobags and similar mattress gabions, preferably filled with wood chips, straw, composts, agricultural waste products, etc., are also particularly useful for building biodegradable erosion control structures, berms, revetments, banks, barriers, dykes, retaining wall structures, channel liners, filter drain systems, etc. for purposes such as mycofiltration and mycoremediation. It will also be noted that heavy cloths may be formed into ‘basket gabions’ which will also provide multiple horizontal layers for growth and routes for vertical colonization when stacked to form revetments, berms, barriers, banks, etc. In general, biodegradable cloths are preferred, but non-biodegradable materials such as plastic polymers may also be inoculated and utilized as an inoculation source for non-sterile bulk substrates. Such mycomats, mycocloths, mycobags and mycogabions may be treated with fungal inocula for immediate use or may be partially overgrown or completely overgrown with fungi and then utilized. In many cases, seeds are also preferably added, such as native grasses, etc. The use of burlap (typically made of jute, flax or hemp) mycobags filled with wood chips on ‘mineral earth,’ the layer beneath topsoil, has also been found to be an effective way to begin the process of soil regeneration.

The use of cardboard, straw, sawdust, etc. layers on top of the inoculated materials (such as bags, blankets, cloths, etc.) or substrate material is useful to ameliorate the loss of water, whether these inoculated materials are overlaid on the ground or buried under wood chips, straw or agricultural waste products. For example, layers of cardboard (top), wood chips (middle), and inoculated cloth or bag (bottom), or alternatively cardboard (top), inoculated cloth (middle) and wood chips (bottom) or variations thereof. The use of moisture retaining materials on top is also useful when ‘sandwich’ layers of inoculated materials and uninoculated substrate are utilized. Ultimately, the insulating material itself will be transformed in a rich soil.

In order to increase fungal penetration of soils, berms, etc. beyond the typical 10-20 cm. (4-8 inch) depth, aeration methods or oxygenated water may be employed. Various methods of aeration and oxygenating water and delivering such will be readily apparent to those skilled in the art. By way of example but not of limitation, water may be oxygenated by means of percolation, high pressure infusion, electrolysis, hydrogen peroxide, chemical reaction, etc.

Where it is desired to use fungally inoculated and enhanced landscaping cloths, mats, gabions, fiber-logs, fiber-bricks or bulk substrates of a size or amount that exceeds even the size of the largest autoclaves (for pressure steam sterilization) or steam pasteurization chambers, or where steam sterilization or pasteurization is not available, the various alternative methods known to the art may be utilized. By way of example but not of limitation, these methods include: 1) Immersion of the landscaping cloth or other substrate in a hydrated lime (calcium hydroxide) solution, thereby largely rendering competitor fungi and bacteria inactive from the drastic change in pH. For example, 2-4 pounds of lime is added for every 50 gallons of water, resulting in a lime/water ration of about 0.5%-1.0%. The cloth or substrate is soaked overnight or for a similar period, the water is drained and the cloth or substrate is inoculated using standard spawn methods or methods as disclosed herein. Such is particularly useful for fungi that can tolerate an alkaline environment better than competitors, such as Pleurotus. Optimizing the parameters for the species being cultivated, such as initial pH of the makeup water, greatly influences the success or failure of this method.; 2) Immersion of the cloth or substrate in a bleach bath utilizing household bleach (typically about 5.25% sodium hypochlorite). For example, 3-4 cups of household bleach is added for every 50 gallons of water, the cloth or bulk substrate is immersed and kept submerged for a minimum of 4 and a maximum of 12 hours, and the bleach leachate is drained off. The cloth or bulk substrate is immediately inoculated.; or 3) Disinfection with hydrogen peroxide (H₂O₂). This technique has been refined by Rush Wayne, who, having become frustrated with the difficulty and expense of creating a sterile environment in his home, refined this technique to a practical level. A full description of this technique can be found at www.members.aol.com/PeroxyMan and detailed instructions may be found in the book Growing Mushrooms the Easy Way: Home Mushroom Cultivation with Hydrogen Peroxide by R. Wayne (1999), Rush Wayne Enterprises, Eugene, Oregon, herein incorporated by reference. It should be noted, however, that much resident contamination can survive this process. While hydrogen peroxide works to kill many fungal spores, yeasts and bacteria by producing a reactive form of oxygen, which destroys cell walls, because fungal compounds have evolved to decompose organic compounds in the environment using peroxides and peroxidases, the mycelia of contaminant fungi and molds is protected from its oxidizing effects. If colonies of mycelium from contaminant fungi have already developed, this method will be of limited advantage. Although not thorough enough to neutralize most of the natural fungi contaminants resident in raw sawdust, straw, etc., hydrogen peroxide can help complete the process started with many preheated substrates. For example, when wood is baked in an oven at 149° C. (300° F.) for 3 hours, compounds are destroyed in the wood that would otherwise neutralize the peroxide. Hydrogen peroxide can be diluted 100-fold, from 3% to 0.03%, into water (less than 60° C. or 140° F.). This water can then drench the substrate to further reduce the likelihood of competitors.; 4) High-pressure extrusion of straw and sawdust and other bulk substrates. This method for treating straw and sawdust utilizes the heat generated from the extrusion of a substrate from a large orifice through a smaller one, producing pellets or a ‘rope’ substrate. The effective reduction of the substrate causes frictional heat to escalate. For example, a 6:1 reduction of straw into a 10 millimeter pellet creates a thermal impaction zone where temperatures exceed 80° C. (176° F.), temperatures sufficient for pasteurization. Alternatively, a roller mechanism may be utilized rather than a narrow orifice, enabling processing of much more substrate mass and producing a matlike product.; 5) The detergent bath method, which utilizes biodegradable detergents containing fatty oils to treat bulk substrates. Coupled with surfactants that allow thorough penetration, these detergents kill a majority of the contaminants competitive to mushroom mycelium. The landscaping cloth, mat or bulk substrate is submerged into and washed with a detergent solution. The environmentally benign wastewater is discarded, leaving the cloth, mat or substrate ready for inoculation.; and 6) A yeast fermentation method may be utilized to render straw and other substrates suitable. Straw can be biologically treated using yeast cultures, specifically strains of bee yeast, Saccharomyces cerevisiae. This method by itself is typically not as effective as those previously described. First, a strain of beer yeast is propagated in 200 liters (˜50 gallons) warm water to which malt sugar has been added (for example, 1-5% sugar broth). Fermentation proceeds for 2 to 3 days undisturbed in a sealed container at room temperature. Another yeast culture can be introduced for secondary, booster fermentation that lasts for another 24 hours. After this period of fermentation, chopped straw or other substrate is forcibly submerged into the yeast broth for no more than 48 hours. Not only do these yeasts multiply, absorbing readily available nutrients, which can then be consumed by the mushroom mycelium, but metabolites such as alcohol and antibacterial byproducts are generated in the process, killing competitors. Alternatively, the natural resident microflora from the bulk substrate may be utilized for submerged fermentation. After 3 or 4 days of room-temperature fermentation, a microbial soup of great biological complexity evolves. The broth, which can be used as a natural biocide, is now removed and the substrate is inoculated. Although highly odiferous for the first 2 days, the offensive smell soon disappears and is replaced by the sweet fragrance of actively growing mycelium. The outcome of any of these alternative methods greatly depends on the cleanliness of the substrate being used, the water quality, the spawn rate, and the aerobic state of the medium during colonization. These alternative methods generally do not result in the high consistency of success (>95%) typical with heat treatment techniques.

It will be noted that normally paper rolls, paper towels, cardboard, etc. are ‘clean’ enough and structurally selectively favors the fungal mycelium so that products constructed of such may be utilized without pasteurization or sterilization (especially cardboard such as corrugated or pressed cardboard).

Where prior sterilization of the ground is desired, the many various methods known to the art may be utilized, for example flame, hydrogen peroxide, hydrogen peroxide/acetic acid, etc.

In another preferred embodiment, fungal inoculum is added to spray hydroseeding equipment or mobile landscaping hydroseeders for delivery of spores and/or hyphae.

Where non-pasteurized or non-sterilized large fabrics or geocloths, including wire mesh reinforced erosion control cloths and synthetic fabrics, are, for example, used for landscaping, used to stabilize soil embankments, slopes and walls, used to promote vegetation growth while providing rockfall protection and/or used for mycofiltration or mycoremediation, a preferred embodiment is ‘spray hydroseeding’ of fungally inoculated products. Spray hydroseeding is performed with a pump for dense liquids, which sprays on to the surface to be greened a mixture consisting of, for example, fungal inocula (spores, dried hyphae, powdered mushrooms, conidia, etc.), seeds, fertilizer if desired, and commercial green hydromulch (a wood fiber mulch) or soil improvement substances, optionally and usually preferably with a binder or tackifier, and water. As an alternative to commercial hydromulch, the numerous other agricultural waste fibers, mulches and composts may be utilized. Such may be preferred to favor the growth of certain species with specialized requirements—for example, Volvariella volvacea, the Paddy Straw mushroom, where rice straw is a preferred substrate. The fungal mycelium which develops after application not only assists the growth of plants and recovery of the ecosystem as above, but also serves to enhance the tenacity of the fabric or geocloth, the many miles of mycelial hyphae forming widespread connections between the cloth and the ground, thus preventing ‘slippage’ and anchoring the fabric cloths, mulch, wood chips, straw, etc.

If desired, the hydroseeding mulch may optionally be partially overgrown or completely overgrown with fungal mycelium prior to use. For example, inoculation and growth for 48 to 72 hours will produce a germinated, actively growing mycelium. Such mulches may be utilized with fresh, actively growing mycelium or may be metabolically suspended via refrigeration, drying or freeze-drying for storage and transport prior to reactivation and use.

A wide variety of landscaping substrates, carriers, products and materials are suitable for practice for the various embodiments of the present invention. Where a bulk substrate mulch is desired, as for example in spray hydroseeding of geocloths utilized to prevent erosion, suitable chopped, chipped, shredded, ground, etc. fiber substrates include by way of example (but not of limitation) woody and non-woody fibers such as wood chips, sawdust, wood pulp, wood mulch, wood wastes, wood pellets and paper fiber pellets, leaf paper, wood-based papers, non-wood papers, pressed cardboard and corrugated cardboard, fiberized rag stock, cellophane, hemp and hemp-like materials, bamboo, papyrus, jute, flax, sisal, coconut fibers and coir, wheat straw, rice straw, rye straw, oat straw and other cereal straws, reeds, rye grass and other grasses, grain hulls and other seed hulls such as cottonseed hulls, cornstalks, corncobs or ground corncobs, soybean roughage, coffee plants, waste and pulp, sugar cane bagasse, banana fronds, palm leaves, the hulls of nuts such as almonds, walnuts, sunflower, pecans, peanuts, etc., soy waste, cactus waste, tea leaves and a wide variety of other agricultural waste products and combinations thereof. Suitable animal fibers include wool, hair and hide (leather) and combinations thereof.

Alternatively, such pressurized spray hydroseeding may be utilized without a cloth for landscaping, agriculture, covering garbage dumps (thus preventing blowing garbage and dispersal by winds and ultimately enabling improved biodegradation of dump materials) and numerous other applications, with the water-fungus-hydromulch mixture being spread over large areas. Such an approach may be preferred where it is desired to avoid the expense of landscaping fabrics or geocloths, the time and effort of installing and securing such fabric blankets, preparation of a relatively smooth surface for installation, etc. The non-fungal component may be varied in the ways known to those skilled in the art to favor the applied fungal species, for example woodland mushrooms, grassland mushrooms, dung inhabiting mushrooms, compost/litter/disturbed habitat mushrooms, mycorrhizal mushrooms, entomopathogenic fungi and combinations thereof.

Using a subset of non-germinating seeds, and/or the outer shells and hulls of germinating seeds within the propelled hydroseed mixture as food, the mycelium can co-exist with germinating seeds in the applied environment, benefiting both, and strengthening ecological fortitude.

Binding agents or “tackifiers” are typically preferably employed as a component of the hydromulch. The tackifier/binding agent component of the mulch enhances the strength and integrity of a mat-like tackified mulch structure and may assist in adhering the mulch structure to the surface upon which it is applied, assisting in the erosion control function and preventing dispersal of the mulch from wind, rain, etc. Various binding agents and tackifiers are known to those skilled in the art; see, for example, U.S. Pat. No. 5,459,181 (1995) to West et al.

For many landscaping and agricultural applications, use of cart-mounted hydroseeding units and the mobile hydroseeding variations will be preferable. Such units are typically utilized to plant lawn grasses, and may be utilized to plant native grasses, wildflowers, mixtures of grasses, shrubs, bushes, trees, crops, etc. if desired. Spores, fresh mycelium, dried or freeze-dried mycelium, powdered mushroom fruitbodies, the many forms of fungi imperfecti and their conidia (asexually produced spores) and related fungal forms and combinations thereof may be easily added to the hydroseeding mixture. Hydroseeding units typically employ mechanical agitation (via paddles or augers inside the tank) or jet mixing (via pump jets) of water and materials; other methods will be readily apparent to those skilled in the art.

Hydroseeding as a fungal mycotechnology works well for numerous reasons. The spores, mycelium or powdered mushroom fruitbodies and the seeds are suspended in a nutrient rich slurry. The contact of the fungal inoculum and seeds with the water triggers the germination cycle of both. The mulch layer seals in the moisture and holds the soil in place (particularly if a tackifier is utilized). The fungal inocula and seed are at an ideal depth for good results. The conditions are right to produce lush growth in a very short time. In addition, such an approach can greatly lower labor costs, with one person simultaneously applying fungal inoculum, hydromulch, seed, fertilizer and tackifier if desired, and water.

For use with trees and other slow germinating plants, a cover crop of, for example, grass seeds or sterile hybrids can be applied in the mixture to give a fast germinating ground cover, the grasses typically germinating first followed by germination of the tree seeds. Alternatively, tree seedlings may be directly utilized. As another example, a cover crop of millet or ryegrass or sterile wheat can also be applied in the mixture to give a fast germinating ground cover until the grass (or native grasses, etc.) being planted becomes established. This method is only recommended for use during the growing season of the particular grass species. Another preferred embodiment utilizes a non-seeding annual grass, with the more expensive non-native grasses being seeded at a later time after the nurturing biosystem has been established.

Another preferred embodiment of the present invention is the use of fungal inocula with agricultural equipment, including planting equipment, harvesting equipment, field preparation equipment and processing equipment with means for delivering fungal inocula. Appropriate methods of modifying agricultural equipment with pumps, sprayers and/or mixers, etc. or of mixing the fungal inocula with seeds (via the slurries above or other means) will be readily apparent to those skilled in the art. Spores, mycelial hyphae and or powdered mushrooms may be introduced into agricultural equipment as liquids, powders, foams, sprays, creams, etc. and combinations thereof or via other methods known to the art so as to provide the benefits of simultaneous inoculation with saprophytic fungi, mycorrhizal fungi, entomopathogenic fungi and/or other beneficial fungi. Alternatively, the fungal inocula may be mixed with seeds and then distributed by the various forms of agricultural planting equipment.

By way of example but not of limitation, such agricultural planting equipment may include seeders, air seeders, planters, air planters, plate planters, vacuum planters, drills, air drills, air seeding systems, row crop cultivators, planting systems, inter-row or between row planting systems, rice transplanters, etc.

Agricultural harvesting equipment may include, by way of example only, combines, round balers, square balers, hay cubers, threshers and threshing machines, forage harvesters, windrowers, rakes, tedders, mowers, rotary mowers, sicklebar mowers, slashers and cutters, straw choppers, stalk choppers, corn pickers, cotton strippers and gins, corn huskers, shellers, rice harvesters, mechanical fruit and nut pickers, loaders, etc. The fungal inocula may be utilized in various manners according to the desired purpose. For example, it may be utilized to inoculate the remaining agricultural waste and/or fields after harvest, thereby providing the numerous advantages discussed herein via inoculation of the agricultural wastes and/or crop fields. Alternatively, the fungal inocula may be utilized to directly inoculate the agricultural products for uses as described herein, for example inoculation of hay or straw with round or square balers, inoculation of hay with tedders, inoculation of grasses with mowers, inoculation of corn husks and corn cobs with huskers and shellers, inoculation of cotton wastes via cotton pickers and strippers, inoculation of cotton seeds and hulls via cotton gins, inoculation via loaders, etc.

In another preferred embodiment, such fungal inocula may be utilized directly with agricultural equipment useful for preparation and/or improvement of fields, orchards, etc. Such equipment includes by way of example sprayers, irrigators, plows, cultivators, air carts, tillers and tillage equipment, disks, openers, rippers, harrows, rotary hoes, blades, flail shredders, flail cutters, rotary cutters, manure spreaders, flame weeders, pruning machines, skids, scrapers, loaders, fertilizer spin spreaders, pendulum spreaders, etc.

In another preferred embodiment, fungal spores and/or mycelium is introduced into shredders and/or chippers to inoculate organic debris laid onto landscapes.

The use of fungal inoculants as described above results in a ‘mycofiltration’ membrane lessening the impact of biological pathogens and chemical pollutants in downstream environments. The fine network of mycelial cells catches bacteria and other biological organisms as well as releasing chemical agents (enzymes, peroxidases and acids) which decompose toxins. In one field experiment, beds of Stropharia rugosoannulata were established on dump truck loads of wood chips in ravines that drained from pastures with a small herd of cattle onto a saltwater beach where oysters and clams were being commercially cultivated. Prior to installing these beds, fecal coliform bacteria seriously threatened the water quality. Once the mycelium fully permeated the sawdust/wood chip beds, downstream fecal bacteria were largely eliminated. The properly located mushroom beds effectively filtered and cleaned the ‘gray water’ runoff of bacteria and nitrogen-rich effluent. This observation was the stimulus for subsequent study by Stamets, Mycofiltration of gray water runoff utilizing Stropharia rugosoannulata, a white rot fungus (1993) (Unpublished Research Proposal awarded a grant by the Mason County Water Conservation District, Shelton, Washington). By using the fungal inoculation mycotechnologies disclosed herein, such as ‘mycocloths,’ ‘mycomats,’ ‘mycobags,’ ‘mycogabions’ and ‘mycoberms,’ such results may be more efficiently and economically accomplished. Such products and methods are in accord with the nature of fungi—riparian habitat buffer zones work primarily because of mycelium. Such colonized mycelial products will thus sequester nitrogen, carbon, phosphorus and other compounds, a novel consequence of actively placing such mycomaterials. Biodegradable mycoberms and similar structures may be built repeatedly over time as an ongoing renewable process.

Such mycelial products are useful for combating virulent bacteria, protists and protozoa, viruses, nematodes, rotifers, etc., for example Escheria coli, Bacillus subtilis, malaria (e.g., Plasmodium falciparum), cholera (Vibrio cholerae), anthrax (Bacillus anthracis), Pfiesteria (Pfiesteria piscicida), a dinoflagellate causing toxic blooms which may assume numerous forms during its lifetime, including a difficult-to-detect cyst stage, an amoeboid stage, and a toxic vegetative stage, water-borne diseases and biological warfare (BW) pathogenic species. Other harmful biological organisms that can be digested and destroyed by fungal mycelia include nematodes, rotifers and insect pests. Thus by infusing mycelium into cloths, rugs, blankets, berms, hydroseeding mulches, soils, etc., targeted disease organisms such as bacteria, fungi, viruses, protozoa, rotifers, amoebas and nematodes can be effectively reduced, ameliorating the downstream impact as well as in residence. Most or all fungi have antibacterial properties; fungi that are preferred for use against bacteria include, for example, Stropharia rugosoannulata, Pleurotus spp. and Fomes fomentarius. F. fomentarius, a mushroom from the old growth forest, produced an army of crystalline entities advancing in front of the growing mycelium, disintegrating when they encountered E. coli, sending a chemical signal back to the mother mycelium that, in turn, generated what appears to be a customized macro-crystal which attracted the motile bacteria by the thousands, summarily stunning them. The advancing mycelium then consumed the E. coli, effectively eliminating them from the environment.

Such an approach may not only combat virulent organisms, but also has the potential to provide fungal products which may be useful in treatment or mitigation of the growth of such diseases. For example, a water extract of Polyporus umbellatus mushrooms obtained from the present inventor (available c/o Fungi Perfecti LLC, P.O. Box 7634, Olympia, Wash. 98507) were found to exhibit 100% inhibition of the growth of Plasmodium falciparum during in vitro assays (Lovy et al., Activity of Edible Mushrooms Against the Growth of Human T4 Leukemic Cancer Cells, HeLa Cervical Cancer Cells, and Plasmodium falciparum, J. Herbs, Spices & Medicinal Plants, 6(4): 49-57 (1999)).

Toxic wastes, contaminants and pollutants that may be remediated by the products and processes of the present invention include, by way of example but not of limitation, organic compounds (taking advantage of the unparalleled ability of fungi to degrade both naturally occurring and synthetic organic molecules), inorganic compounds, and biological contaminants including living organisms such as bacteria, viruses, protists, nematodes, rotifers and combinations thereof.

More specifically, by way of example only, such organic compounds include hydrocarbons such as polynuclear aromatic hydrocarbons (PAHs), cyclic hydrocarbons and hydrocarbon chains such as alkanes and alkenes, including the components of lubricants, fuels and solvents and additives such as methyl t-butyl ether (MTBE), fertilizers, chemical pesticides including organophosphate pesticides and organochlorines such as DDT (dichlorodiphenyltrichloroethane), chlordane and toxaphene, the many dioxins such as 2,3,7,8-tetrachlorodibenzo-p-dioxin (TCCD) and related furans, organochlorines and organobromides such as pentachlorophenol (PCP), polychlorinated biphenyls (PCBs) and polybrominated biphenyls (PBBs), nitrogenous compounds such as such as ammonium nitrate, urea, purines and putriscines, chemical warfare (CW) agents and nerve gases such as the organophosphates Sarin (GB or O-isopropyl methylphosphonofluoridate), Soman (GD or pinacolyl methylphosphonofluoridate), Tabun (GA or O-ethyl N,N-dimethylphosphoramidocyanidate), VX (O-ethyl S-[2-diisopropylaminoethyl] methylphosphonothiolate) and VX family compounds, and their surrogates such as isopropyl methylphosphonic acid (IMPA) and dimethyl methylphosphonate (DMMP), and combinations thereof. One polypore mushroom in the inventor's culture collection destroys the core constituent base of the toxic nerve gas agents VX and Sarin. The fungi are also useful for remediation of explosives (such as gunpowder and trinitrotoluene (TNT)), explosive residues and explosives manufacturing byproducts (such as dinitrotoluene (DNT)). By using cold-weather fungal strains, temperature-sensitive munitions can be decomposed without the dangerous heat build-up associated with typical compost mycoflora. Other contaminants that may be remediated by the present invention include by way of example creosote, alkaloids such as caffeine, endocrine-disrupting compounds such as estradiol, steroids and other hormones, pro-hormones or hormone-like compounds, detergents and soaps, textile dye pollutants including aromatic dyes, medical wastes, urban runoff, industrial wastes and the many other toxic or unpleasant byproducts of human activities. Such fungal products infused with fungi capable of decomposing biological and chemical warfare toxins and industrial toxins can be used to decontaminate toxic landscapes, battlefield and otherwise, thus leading to reuse of valuable land.

One preferred type of fungal blanket, mat, bag or gabion is designed specifically to treat oil spills and slicks. The mycomaterial is preferably made of adsorbent biodegradable fiber materials and inoculated with spores and/or hyphae of oil-eating fungi. Thus the oil is soaked up by the mat material and digested by the mycelium of the fungus. A strain of Pleurotus ostreatus has proven particularly effective in digesting and breaking down petroleum oils (PAHs and alkanes); other preferred species include, by way of example but not of limitation, Trametes versicolor, Ganoderna lucidum and other fungal species as listed below. For soaking up and bioremediating spills on ocean beaches, salt-water marsh fungi are typically preferable, for example Psilocybe azurescens, Psilocybe cyanescens and Flavodon flavus.

Phosphorylated compounds such as the chemical warfare gases and many organophosphate pesticides have proven particularly resistant to breakdown and bioremediation, as few organisms are equipped with the appropriate dephosphorylating enzymes. Fungi, on the other hand, have a number of enzyme systems and paths for dealing with phosphorylated compounds and are therefore particularly suited for remediation of organophosphates. Preferred species include polypore fungi such as Trametes versicolor, Fomes fomentarius, Fomitopsis officinalis, Fomitopsis pinicola, Phellinus igniarius, Phellinus linteus and the other polypores listed below, agarics such as Psilocybe azurescens and Psilocybe cyanescens containing phosphorylated tryptamine compounds and their dephosphorylated analogs, luminescent fungi utilizing adenosine triphosphate, luciferin and luciferase for bioluminescence, and other phosphorus-rich mushroom fungi such as Agrocybe arvalis, Collybia (C. tuberosa and C. albuminosa), Coprinus comatus, Lycoperdon perlatum and L. lilacinum, Pleurotus species, esp. P. ostreatus and P. tuberregium and Psathyrella, i.e. P. hydrophila. Combinations may be preferred in certain applications as bringing a broad range of phosphorus related enzymes to bear.

Since both Psilocybe azurescens and Psilocybe cyanescens can possess up to 1-2% psilocybin, a phosphorus rich molecule, and/or psilocin, the product of dephosphorylation of psilocybin, these species can be used to dephosphorylate toxins wherein phosphorus contributes to the toxicity of the pollutant (such as the phosphorylated chemical warfare gases above and organophosphate pesticides). Grassland species such as Psilocybe semilanceata, also rich in psilocybin, may also be preferably employed; such grassland species have the advantageous characteristic of acting as saprophytes, decomposing organic matter, or acting as ectomycorrhizal species, directly benefiting plants via symbiosis, depending upon circumstances. The non-psilocybin producing Blue Stropharia (blue-staining) species can also be phosphorus containing and equipped with dephosphorylating enzymes. These species include Stropharia aeruginosa, S. cyanea, S. albocyanea and S. caerulea, and may be substituted where laws restrict the use of the psilocybin-positive species, as may non-psilocybin containing blue-staining Panaeolus, Conocybe, Gymnopilus, Inocybe and Pluteus. Alternatively, specific enzyme blockers and/or other agents that block the biosynthetic pathway of psilocybin and psilocin may be utilized. In another approach, the Psilocybe species, which are known to take up substituted tryptamines and convert them to non-naturally occurring analogs of the natural tryptamine products, may be fed a substituted tryptamine that would, on 4-hydroxylation or phosphorylation, produce an inactive compound. Such substitution may be in the 4- position or in the 2-, 5-, 6-, N-, alpha-, etc. positions or combinations thereof. Such substituted tryptamine analogs may thus block or overwhelm the natural enzymes and phosphorus compounds. Similarly, the phosphates such as organophosphate pesticides or nerve gases may be used to overwhelm the naturally occurring enzymes to the exclusion of naturally occurring psilocybin and psilocin. As another alternative, non-fruiting strains of Psilocybe may be selected. As yet another alternative, Psilocybe strains may be used solely in a mycelial state prior to the production of psilocybin and psilocin—for example, it has been found with Psilocybe cyanescens that no psilocybin or psilocin is formed in pre-primordial mycelium, the mycelium knot stage of the mushroom being the earliest stage at which psychoactive compounds could be detected. Gross, J. Forensic Sci., 45(3): 527-37 (May 2000).

Luminescent mushrooms such as Armillaria mellea, A. gallica, A. bulbosa, Mycena citricolor, M. chlorophos, Omphalotus olearius (Clitocybe illudens) and Panellus stypticus present another example pathway of phosphorus utilization by fungi that may be combined with the non-luminescent species. Like the firefly and other organisms, fungi may exhibit bioluminescence involving enzymatic excitation of a molecule to a high-energy state and return to a ground state, accompanied by the emission of visible light. Important molecular components are luciferin, a heat-stable heterocyclic phenol and luciferase, a heat-labile enzyme. Luciferin and ATP are thought to react on the catalytic site of luciferase to form luciferyl adenylate, which is oxidized by molecular oxygen to yield oxyluciferin, which emits light on returning to the ground state. A peroxide is presumed to be formed as an intermediate.

The growth of algae in ponds and lakes can be directly attributed to the phosphorus-rich runoff from agricultural fertilizers and other industrial pollutants. Phosphorus is typically the ‘limiting nutrient’ of algae growth. By removing phosphorus using mycocloths, mycomats and mycoberms infused or spray hydroseeded with dephosphorylating fungi such as Trametes versicolor, Psilocybe azurescens, and others, the over-growth algae can be limited in lakes and ponds, providing cost and ecological saving benefits to fishery ecologies and the watershed. A similar approach may be employed in those soils and waters contaminated with organophosphate pesticide residues. Floating mats of biodegradable materials may be infused with the mycelia of anti-microbial fungi such as Fomes fomentarius, Fomitopsis officinalis, Ganoderma applanatum, Ganoderma oregonense, Trametes versicolor, Lentinula edodes, Laetiporus sulphureus, Pleurotus eryngii, Pleurotus ostreatus, Polyporus umbellatus, Psilocybe semilanceata, Schizophyllum commune, Stropharia rugosoannulata, and Calvatia species and placed into aquatic systems such as, but not limited to, ponds, lakes, streams, rivers, and ditches for an effective treatment in reducing waterborne disease microbes including but not limited to Escherichia coli, Plasmodium falciparum, Streptococcus spp., Staphylococcus spp., Listeria spp., Yersinia spp., Shigella spp.) and parasites (e.g., Giardia spp.)

Inorganic contaminants that may be remediated by fungi include by way of example metals, phosphates, sulfates, nitrates, radionuclides and combinations thereof. The fungal mycelia may or may not be able to chemically alter an inorganic contaminant, for example metals or radionuclides. However, the inorganic contaminant may be concentrated from the surrounding ecological environment into fruiting bodies of the fungi. With mixed organic/inorganic contaminants such as organometallic compounds, the fungi may both degrade the compound and concentrate the metal component.

The ability of higher fungi to concentrate heavy metals, metabolize phosphorus compounds, etc., combined with the novel fiber products and methods of the present invention allows use of fungally impregnated materials, within or in absence of a matrix of biodegradable or non-biodegradable materials, to sequester and concentrate heavy metals, radioactive or otherwise, which then can be removed to eliminate toxins topically and subsurface. Metallic effluents and ores may be treated with specifically targeted fungi, for example the phosphate remediating mushrooms for phosphate ores and runoff and/or metal concentrating mushroom fungi. In addition, the fungi may favorably metabolize the organic portion of organometallic compounds via mycofiltration and mycoremediation.

Such residual organic debris from mycelia and the delivery systems herein could be economically or profitably separated from the metals through incineration, biodigestion with other organisms such as bacteria, protozoa, yeasts, and/or via chemical treatments including acids, enzymes and catalysts, including also the many other approaches known to the art. Such an approach can also be favorably employed to control metal-laden runoff from gold mines, silver mines, uranium mines, etc., providing control of mine wastes while concentrating the valuable residual metals. Once sequestered and concentrated, the metals may be removed by mechanical, chemical and/or biological means. A number of mushroom fungi are known to concentrate metals, including various edible mushrooms. One family of preferred genera is Collybia and the similar Marasmius and their numerous “satellite genera” in this “taxonomically troubled” group. Such satellite genera (Collybia ‘sensu lato’) include Caulorhiza, Oudemansiella, Flammulina, Crinipellis, Callistosporium, Micromphale and Marassmiellus.

Examples of previous methodologies include those disclosed in U.S. Pat. No. 5,021,088 (1991) to Portier for separation and recovery of gold and U.S. Pat. No. 4,732,681 (1988) to Galun et al. for methods and systems for use of a strain of Cladosporium cladosporioides to decrease heavy metal concentrations such as lead, zinc, cadmium, nickel, copper and chromium in industrial effluents. These and other similar methods may optionally be combined with the higher fungi and the present invention for improved separation and recovery from carbonaceous or pyritic or phosphate ores and combinations thereof, including both gold and non-gold heavy metals such as the radioactive and toxic metals. Thus the ore or industrial effluents containing the various heavy metals may be treated with microorganisms, such as fungi imperfecti and/or autotrophic bacteria such as Thiobacillus ferroxidans and T. thlooxidans, to leach soluble iron, copper and other metals and sulfuric acid via oxidation of iron and sulfur prior to treatment with the delivery systems of the present invention.

U.S. Pat. No. 4,021,368 (1977) to Nemec et al. discloses use of lower fungi microorganisms combined with polymers to “stiffen” the fungus and eliminate the typical problems arising from fungi in general having a low long term mechanical rigidity, causing difficulties in retention or absorption. A stiff, coherent mycelial mat as provided by the delivery systems of the present invention would be advantageous for collection of metal-enriched mycelium and or mushrooms. Such may be provided via the present invention in the form of a landscaping blanket, rug or mat or via bags or gabions or via hydroseed fungal inoculation, optionally reinforced by a polymer, metal or biodegradable fiber or combination thereof or other support, with or without barrier materials ranging from tarps to complex barriers . Alternatively, such supports and/or barriers may be utilized with spray hydroseeding of hydromulch, wood chips, straw, etc., optionally with tackifier, with ‘sandwich’ inoculation if desired, with or without fiber cloths or gabions or such, so that the fungal species form a coherent, matlike mycelium. Such an approach is also useful for biological concentration of ores, ore slurries, etc., particularly of the heavy metals, as well as the various other applications disclosed herein for mycoremediation, mycofiltration, mushroom and plant cultivation, etc.

With or without such treatment with lower fungi and/or bacteria, mine waste, effluent or ore substrate can be inoculated with saprophytic mushrooms known for high yields, thereby allowing for the further concentrating and sequestering of precious metals, toxic metals such as lead, and/or the radioactive metals, both toxic and precious. For instance, Oyster mushrooms, Pleurotus ostreatus, commonly convert 10% of the dry mass of the substrate into dried mushrooms, allowing for a ‘harvested’ crop which can be efficiently removed from the background environment. Subsequent to Oyster mushrooms ceasing flushes, another species of mushrooms can be introduced, such as Stropharia rugosoannulata, which can further concentrate the targeted compounds. Another round of concentration may be carried out at that point by the numerous mushrooms which will grow upon the rich soil that has been created via lignin degradation, including mushrooms such as the ‘Shaggy Mane,’ Coprinus comatus, and the wide variety of mushroom species ranging from gourmet lawn and field mushrooms to little brown mushrooms to ‘poisonous to humans’ mushrooms. By sequencing accumulator and hyperaccumulator mushroom species, progressively greater extraction and/or concentration of valuable metals can be achieved.

The fungal delivery systems of the present invention may also be favorably combined with the techniques of phytoremediation (bioremediation via plants) for maximum effectiveness of bioremediation of metals, persistent organics, chlorinated organics, organophosphates, etc., including those 400+plants that have to date been found to be “hyperaccumulators” of metals, chlorinated solvents, etc. Suitable phytoremediation techniques for optional combination with the delivery systems of the present invention include phytoextraction (phytoaccumulation), rhizofiltration, phytostabilization, phytodegradation (phytotransformation), rhizodegradation (enhanced rhizosphere biodegradation), phytostimulation, or planted-assisted bioremediation/degradation), and phytovolatilization. It is thought by the present inventor and others that fungi assist and enable successful and efficient hyperaccumulation via various direct and symbiotic mechanisms.

The present inventor has observed that one such preferred hyperaccumulator species, the hybrid poplar, does particularly well in the presence of saprophytic, wood decomposing mushrooms on wood chips and fibrous media placed above the soil. By way of example only, hyperaccumulator species for organics include poplars, cottonwood, mulberry, juniper, sunflowers, fescues, ryegrasses and other grasses, clover, Indian mustard, duckweed, parrotfeather, etc. and combinations of these and the numerous other hyperaccumulators and accumulators found in the plant world. Such hyperaccumulator species are, by way of example only, able to extract and detoxify chlorinated solvent such as methylene chloride and trichloroethylene (a major groundwater pollutant) and trinitrotoluene (TNT) via the phytoremediation mechanisms as well as providing the known admirable habitat improvement properties of healthy trees and plants via shade, shelter, humidity maintenance, provision of lignin for conversion by fungi into nutrients, etc.

In a preferred embodiment, poplars and other hyperaccumulator trees, in symbiosis with fungi, display and maintain hydraulic control—mature poplars have been estimated to transpire between 50 and 300 gallons of water per day out of the ground. Hydraulic control is the use of plants to rapidly uptake large volumes of water to contain or control the migration of subsurface water. The water consumption by the poplars and other trees decreases the tendency of surface contaminants to move towards ground water and into drinking water. There are several applications that use plants for this purpose, such as ‘riparian corridors’ or ‘buffer strips’ and ‘vegetative caps.’ Banks of poplars have also been used to stabilize petroleum-contaminated groundwater flow, since the tree's prodigious transpiration rate prevents movement of groundwater off site. The same poplar technique has been shown to be an effective way to keep agricultural runoff from entering streams, lowering pesticide and fertilizer contamination of waterways, and thus may be favorably and advantageously combined with the delivery systems and mycofiltration techniques of the present invention which are separately able to perform large scale mycofiltration and mycoremediation.

Hyperaccumulator plants are known in the scientific research and patent literature that can concentrate metals thousands of times above normal levels and can optionally be combined with the fungal delivery systems for mine effluents and metallic ores described herein. For example, planted on soil laden with nickel, Streptanthus polygaloides of the cabbage family accumulates nickel up to one percent of its dry weight in its leaves and flowers. Detoxifying the soil is as simple as harvesting the plants. The ‘brake fern’ (Pteris vittata) hyperaccumulates arsenic from contaminated soil, attaining concentrations of arsenic as much as 200 times higher in the fern than the concentrations in contaminated soils where it was growing. It will accumulate arsenic even from soils having normal background arsenic levels. As another example, after concentration and chelation via addition of a chelating agent (or chelation and subsequent biological availability by the present invention), lead can be accumulated by Indian mustard (Brassica juncea). Indian mustard, in addition to lead, will hyperaccumulate chromium, cadmium, nickel, selenium, zinc, copper, cesium, and strontium. Sunflowers are known to absorb radioactive cesium and strontium, although much of the metal remains bound in the root system, making it a poor candidate for soil cleanup. After the 1986 Chernobyl nuclear disaster, Ilya Raskin suspended sunflowers from Styrofoam rafts in ponds, where they thrived, concentrating the metals up to 8,000 times the level in the water itself, removing between 90 and 95 percent of the radioactivity from the pond. The plants are removed, dried, and disposed of as radioactive waste. In combination with the delivery systems of the present invention, hyperaccumulators may optionally be employed with the fungal keystone species, organic and inorganic nutrient gathering fungal species, and/or metal concentrating fungal species and delivery systems of the present invention.

Whereas the literature of phytoremediation often teaches away from use of fungi with plants or teaches the use of nutrient poor or nutrient limited soils for some applications, often leading to poor hyperaccumulator growth, such will typically not be the case when practiced with the present invention, with or without added plant hyperaccumulators, as the fungi introduced by the delivery systems herein tend to function as keystone species, leading to lush habitats and vigorous growth of all plants, including hyperaccumulators, with ecosystems better able to function as bioremediation agents.

Such fungally colonized mycelial products protect sensitive watersheds such as salmon spawning grounds, providing mushroom and mycelial biomass which then feed developing larvae of numerous insects which benefit fisheries through enhancement of the food chain and from protection from upland runoff. The present invention provides further advantages in providing mycofiltration of pesticides, including both organophosphate and halogenated pesticides, which are thought in minute quantities to interfere with salmon's olfactory sense, thereby impeding the return to breeding grounds and successful reproduction. Also provided are the sediment and silt filtering advantages of mycofiltration. Sediment and silt runoff into salmon and trout spawning grounds are know to create environment hostile to egg survival. Similar negative habitat effects result from runoff into other bodies of water. By utilizing mycofiltration, the silt and sediment becomes part of a rich soil as opposed to a marine pollutant. The present invention as described herein may be effectively employed to reduce, ameliorate, limit or prevent the impact of pesticides and other agricultural and/or urban contaminants upon riparian habitats and marine environments and the associated fisheries, recreational use, drinking water, etc.

Fungi also present novel advantages in sequestration of carbon. The international Kyoto Accords of 1998 helped establish a carbon-credit system, an incentive-based system wherein those countries sequestering carbon, effectively reducing the release of carbon dioxide, are rewarded. The concern is to lessen the ‘greenhouse effect’, a major factor in global warming.

The no-till method of farming, wherein stubble is left for natural decomposition, sequesters carbon in the soil. A study by Hu et al., “Nitrogen limitation of microbial decomposition in a grassland under elevated C0₂,” Nature, 409: 188-191 (11 Jan. 2001), shows that elevation of carbon dioxide levels in grasslands reduces microbial activity, specifically as seen through the metabolism of nitrogen. Hence as C0₂ goes up, microbial activity goes down. What these and other researchers have not yet recognized is that the mycelium can intelligently regulate their grow-rates and out-gassing to normalize the gaseous environment of the ecosystem in which they grow. The cellular architecture of the fungal mycelial networks is made of carbon-heavy molecules (chitin, carbohydrates and polysaccharides) and hence habitats infused with mycelium using the present invention significantly enhance their value in terms of augmented carbon credits.

In actively restoring devastated habitats using fungally impregnated biodegradable materials, the current invention relies on the naturally gas-governing properties of the selected fungal species. Encouraging the growth of mycelium, and selecting the constellation of fungal species target-specific to the toxic or threatened landscapes, enormous amounts of carbon can be sequestered by the exoskeleton of the mycelial network, heavy in carbon-rich molecules such as chitin and polysaccharides, and/or through the protein-rich contents of the internal cell components. Furthermore, the active placement of mycelial mosaics in a habitat additionally sequesters carbon directly external to its cellular architecture through the production of extracellular enzymes which convert cellulose precursor compounds into arabinoxylanes and arabinogalactans. Mycelial mats of saprophytic and other fungi may cover areas ranging from small plots to thousands of acres. The mushroom mycelial mat is in fact a carbon bank.

The carbon credit system can also be economically applied when incorporating the use of mycelium into organic debris fields and mycomats in the reclamation of roads back into native ecosystems, optionally applying the phytoremediation approaches above. Thousands of miles of roads must be returned to natural conditions and the current energy crisis has caused ‘hog fuel’ (=chipped junk wood used for furnaces) to skyrocket. The loss of carbon from the ecosystem is an unfair economic practice as the hog fuel prices are not being valued for their inherent carbon value. As governments incorporate/recognize that the value of wood debris also should be considered in terms of carbon credits, then the cost of using mycomats can be justified as an economically valuable, cost-effective product and procedure for incorporating carbon dioxide into fungi and plants in both microsphere and biosphere.

Hence a major advantage of this invention is the active prevention of atmospheric carbon dioxide through sequestering of carbon into the mycelial network within the soil matrix. Thus, fungal growth can ‘bank-roll’ the carbon credit system through such examples as the ‘no-till’ method and/or through repairing threatened ecosystems by designing the insertion of keystone fungi most beneficial to targeted environmental goals. By sequestering carbon and increasing the value of the carbon credit, the mycotechnologies of the present invention provide not only a cost effective method, but also the numerous advantages arising from habitat improvement.

Such landscaping substrates, cloths, carrier products, hydroseeding equipment and agricultural equipment also provide means of introducing mycorrhizal fungi. Such mycotechnologies also provide means for introduction and “companion cultivation of saprophytic mushrooms” with agricultural crops. The benefits of mycorrhizal fungi are well known; the present inventor and others have also found that companion cultivation of saprophytes enhances both quantity and quality of yields of grains and vegetables and other crops. As mycelia bind soil particles (aggregation), soil compaction is decreased and aeration is increased, allowing roots, oxygen, carbon dioxide and water to move through the soil. This improvement in soil quality may be noticed as a ‘bounce factor’ when walking over soils inoculated with saprophytic fungi. For example, Hypsizygus ulmarius on sawdust, covered with straw, has been found to be of great benefit to many crops and plants, including corn, beans and Brussels sprouts; large ears of corn were produced in a poor experimental soil, whereas previously the present inventor had not been able to successfully cultivate corn in his garden due to growing season and climate limitations. Hypholoma sublateritium was also of great benefit to corn cultivation. Stropharia rugosoannulata is known to benefit corn and was found to provide such a benefit, particularly in the second and following years after inoculation. Thus companion cultivation of saprophytes also offers preferred methods of improving crop yield while reducing the need for fertilizers. See Pischl, C., Die Auswirkungen von Pflanzen-Pilzmischkulturen auf den Bodennaehrstoffgehalt und die Emteertraege (1999), Master's Thesis, Leopold-Franzens-Universitat Innsbruck. Mushrooms were observed fruiting underneath seedlings, the dewdrop formation and drip zone providing a preferred fruiting site. However, the plants and mushroom species must be carefully matched: while the Oyster-like mushroom Hypsizygus ulmarius had a beneficial effect on some neighboring crop plants, the Oyster mushroom Pleurotus ostreatus did not (Pischl, 1999). On the other hand, for nematode infested soils, P. ostreatus and other Pleurotus species may be preferred, the mycotechnologies herein acting as a nematode-control delivery system.

Inoculation of sawdust, straw or other fiber substrates placed on top of the soil has been found by the present inventor to be superior to and generally preferred to methods of inoculating and mixing with the soil for agricultural purposes; a more beneficial microclimate, microflora and biosphere results from placement of inoculated wood, straw, etc. on top of the soil. The no-till practice in particular improves the soil quality by fostering saprophyte populations that enhance the formation of water stable aggregates, thereby improving aeration, water infiltration, water retention and plant nutrient reserves. Such an approach also has the potential for producing gourmet and medicinal mushrooms.

The use of fungi (mycorrhizal and symbiotic saprophytic fungi) in a biodegradable matrix further aids the growth of resident and implanted flora. Such examples include, but is not limited to the enhancement of native or erosion-control grasses whose growth is enhanced from the fungal components described herein. As the organic structural matrix, for example, a straw/coconut cloth, is decomposed by the fungal component, grasses benefit from the newly available nutrients liberated by the mycelium, from the protective effect of the selected mycelium against invasive pathogenic fungi and bacteria, and from the increase in water retention in otherwise porous (sandy) soils. In both natural and man-made habitats, the introduction of these fungi is an active component in enhancing environmental health. For instance, the tenacity of Ammophila maritima, a dune grass planted by the Army Corp of Engineers to prevent jetty erosion around the Columbia River as it enters the Pacific Ocean, is significantly enhanced through the domination of the mycelium of Psilocybe azurescens and P. cyanescens in the top soils of that biosphere.

Of particular use where insect pest control is desired are the entomopathogenic fungi Metarhizium, Beauveria, Paecilomyces, Verticillium, Hirsutella and Cordyceps, either as the sole fungal species or in combination with saprophytic and/or mycorrhizal species. In addition to known uses of spores, the preconidial mycelium of entomopathogenic fungi has been found to be attractant and/or pesticidal to such pest insects as termites, fire ants, carpenter ants, etc. See U.S. Pat. No. 6,660,290 (2003) for MYCOPESTICIDES and U.S. patent application Ser. No. 09/969,456 (2001) for MYCOATTRACTANTS AND MYCOPESTICIDES, herein incorporated in their entirety by reference. Extracts of the pre-conidial mycelium of entomopathogenic fungi, for example extracts of Metarhizium, Beauveria and/or Cordyceps, are also useful for attracting and/or killing insects and may be favorably combined with the fungal delivery systems disclosed herein. See MYCOATTRACTANTS AND MYCOPESTICIDES above.

Insect pest control benefits are also provided by mycorrhizal fungi. Plants infected by endophytic fungi are known to be chemically protected against consumption by insect pests, for example aphids. Insect herbivore-parasite interaction webs on endophyte-free grasses show enhanced insect abundance at alternate trophic levels, higher rates of parasitism and increased dominance by a few trophic links, whereas plants infected with endophytes alter insect herbivore abundance, selectively favoring beneficial insects and higher organisms. It is conceivable that the effect of plant endosymbionts on food webs will cascade up through various trophic pathways and can mediate competitive interactions between plant species affecting vegetation diversity and succession. Ornacini et. al., Symbiotic fungal endophytes control insect host-parasite interaction webs, Nature, 409: 78-81 (4 Jan. 2001). Thus in addition to their direct symbiotic effects benefiting plants, it is expected that mycorrhizal fungi can reduce pest insect herbivores, thus favoring beneficial insects and higher organisms and thereby increasing biodiversity.

The parasitic fungi are particularly useful for the control and extermination of invasive plant species, for example, the Melaleuca trees in the Everglades. Such parasitic fungi include, for example, Phellinus weirii and Armillaria mellea, two aggressive species. By use of non-sporulating strains (as have been developed for Pleurotus ostreatus) incorporated into mycocloths or hydroseed spray, undesirable cross-infection outside of the targeted area can be limited.

Control of plant pathogens such as Rhizoctonia solani, Sclerotium rolfsii, Verticillium dahliae and other soilborne plant diseases may also be provided by saprophytic and mycorrhizal fungi and by fungi imperfecti such as Trichoderma viride, T. harmatum and Gliocladium virens.

Such mycotechnologies may be beneficial not only on Earth, but also eventually in aiding the establishment of habitats in space colonies and in the colonization of other planets. Such fabrics could be bio-engineered from planetary surface dust (‘soils’) and impregnated with spores of fungi and other organisms. Since there can be more than a billion spores per gram, spores can be economically transported via drone or spaceship to the targeted planetary body or space station. Their low weight/mass makes them economically attractive bio-cargo for transportation through interplanetary and interstellar space and the importance of fungi as a keystone species makes them essential in any self-sustaining habitat.

Water and/or oils are preferably used to deliver spores and mycelial hyphae, although spores and/or mycelium may be applied directly to the landscaping materials, or traditional inoculation methods with grain and/or sawdust spawn, etc. may be utilized (see Stamets, Growing Gourmet and Medicinal Mushrooms (1993, 2000) and Stamets et al., The Mushroom Cultivator (1983), both herein incorporated by reference). Petroleum oils can be readily digested by certain fungi and biodegradable oils are readily digested by most or all fungi perfecti and fungi imperfecti. Therefore oil-spore or oil-hyphae mixtures or water-oil-spore or water-oil-hyphae suspensions, with or without seeds, provide an alternative to the water-spore or water-hyphae slurries which may be utilized in the practice of the present invention. In general, where oils are utilized, biodegradable oils are preferred as offering a more readily available nutritional source to a wide variety of fungi. However, as some strains of white rot fungi have proved to be voracious consumers of petroleum oils, species of oil-eating fungi may be utilized with petroleum and mineral oil lubricants and synthetic and semi-synthetic lubricants, as well as with biodegradable lubricants, vegetable oil lubricants, modified vegetable oil lubricants, animal lubricants and combinations and blends of these lubricants. Numerous vegetable oils are suitable, including by way of example canola, rapeseed, castor, jojoba, lesquerella, meadowfoam, safflower, sunflower, crambe, hemp, flax, cottonseed, corn, olive, peanut, soybean and other such vegetable oil sources. Such spored or hyphal oils may also be preferably employed in applications such as ecological rehabilitation, mycoremediation and mushroom growing where use of an oil as an additional nutritional source is desired.

The spores or fungal hyphae transfer agents may optionally contain further amendments including germination enhancers, growth enhancers, sugars, nutritional supplements, surface active and wetting agents, spore and hyphae encapsulating materials, yeasts, bacteria, fungi imperfecti, etc. Fungal hyphal mass can optionally be dried or freeze-dried and packaged, with or without additional spores, in spoilage-proof containers for marketing to end users as a seed and slurry additive. Fresh mycelial hyphae or mycelial mass is best used immediately rather than stored for long periods.

Information on gathering useful and beneficial mushrooms for spores or hyphae may be found in standard mycological field guides such as Mushrooms Demystified (1979, 1986) by David Arora and The Audubon Society Field Guide to North American Mushrooms (1981, 1995) by Gary Lincoff.

As one gram of spores of, for example, Ganoderma lucidum may contain more than a billion spores, it is therefore a simple matter to mix an effective amount of spores into water or oil using mechanical or manual mixing techniques known to the art and thereby provide a large number of potential inoculation points.

Fungal spores may gathered via a variety of means, including but not limited to large scale spore-printing on surfaces and collection from fresh and/or dried mushrooms. A unique method developed by the present inventor is to collect spores from the flexible poly-tubing or other ducting used for distributing air within mushroom growing rooms and mushroom farms. This method is efficient in gathering substantial spore mass.

Mycelial hyphae (including mushrooms, a form of mycelial hyphae) may be cultured using standard mycological techniques for mushrooms. Further information on techniques suitable for production of many of the preferred gourmet, medicinal and ecorestorative mushrooms and their spores and mycelial hyphae may be found in applicant's books, Growing Gournet and Medicinal Mushrooms and The Mushroom Cultivator, supra. One cost-efficient method for expansion of mycelial mass for small-scale practice of the present invention are commercial aerobic compost tea fermentors, which allows growers to culture a very high concentration of aerobic microorganisms in approximately 24 hours utilizing fine air particles infused into the tea.

Virtually all fungi may be useful in habitat preservation and restoration, reforestation and agriculture. Fungi useful in the present invention include saprophytic fungi (including gilled, polypore and other types of mushrooms), mycorrhizal fungi (which form a mutually dependent, beneficial relationship with the roots of host plants ranging from trees to grasses to agricultural crops, as may certain saprophytic fungi), and fungi imperfecti (those asexually reproducing fungi related to the sexually reproducing “fungi perfecti” or “mushroom fungi”). All fungi and their spores and hyphae should be considered to be a useful part of the invention.

Suitable fungal genera include, by way of example but not of limitation, the gilled mushrooms (Agaricales) Agaricus, Agrocybe, Armillaria, Clitocybe, Collybia, Conocybe, Coprinus, Flammulina, Giganopanus, Gymnopilus, Hypholoma, Inocybe, Hypsizygus, Lentinula, Lentinus, Lenzites, Lepiota, Lepista, Lyophyllum, Macrocybe, Marasmius, Mycena, Omphalotus, Panaeolus, Panellus, Pholiota, Pleurotus, Pluteus, Psathyrella, Psilocybe, Schizophyllum, Sparassis, Stropharia, Termitomyces, Tricholoma, Volvariella, etc.; the polypore mushrooms (Polyporaceae) Albatrellus, Antrodia, Bjerkandera, Bondarzewia, Bridgeoporus, Ceriporia, Coltricia, Daedalea, Dentocorticium, Echinodontium, Fistulina, Flavodon, Fomes, Fomitopsis, Ganoderma, Gloeophyllum, Grifola, Hericium, Heterobasidion, Inonotus, Irpex, Laetiporus, Meripilus, Oligoporus, Oxyporus, Phaeolus, Phellinus, Piptoporus, Polyporus, Schizopora, Trametes, Wolfiporia, etc.; Basidiomycetes such as Auricularia, Calvatia, Ceriporiopsis, Coniophora, Cyathus, Lycoperdon, Merulius, Phlebia, Serpula, Sparassis and Stereum; Ascomycetes such as Cordyceps, Morchella, Tuber, Peziza, etc.; ‘jelly fungi’ such as Tremella; the mycorrhizal mushrooms (including both gilled and polypore mushrooms) and endomycorrhizal and ectomycorrhizal non-mushroom fungi such as Acaulospora, Alpova, Amanita, Astraeus, Athelia, Boletinellus, Boletus, Cantharellus, Cenococcum, Dentinum, Gigaspora, Glomus, Gomphidius, Hebeloma, Lactarius, Paxillus, Piloderma, Pisolithus, Rhizophagus, Rhizopogon, Rozites, Russula, Sclerocytis, Scleroderma, Scutellospora, Suillus, Tuber, etc.; fungi such as Phanerochaete (including those such as P. chrysosporium with an imperfect state and P. sordida); the fungi imperfecti and related molds and yeasts including Actinomyces, Altemaria, Aspergillus, Botrytis, Candida, Chaetomium, Chrysosporium, Cladosporium, Cryptococccus, Dactylium, Doratomyces (Stysanus), Epicoccum, Fusarium, Geotrichum, Gliocladium, Humicola, Monilia, Mucor, Mycelia Sterilia, Mycogone, Neurospora, Papulospora, Penicillium, Rhizopus, Scopulariopsis, Sepedonium, Streptomyces, Talaromyces, Torula, Trichoderma, Trichothecium, Verticillium, etc.; and entomopathogenic fungi such as Metarhizium, Beauveria, Paecilomyces, Verticillium, Hirsutella, Aspergillus, Akanthomyces, Desmidiospora, Hymenostilbe, Mariannaea, Nomuraea, Paraisaria, Tolypocladium, Spicaria, Botrytis, Rhizopus, the Entomophthoracae and other Phycomycetes, and Cordyceps. It will be noted that some entomopathogenic fungi imperfecti and molds can go through a perfect stage, with the perfect form often getting a new name. It will also be noted that such fungi imperfecti, molds and yeasts may produce spores, conidia, perithecia, chlamydospores, etc. and other means of generating progeny. All such fungi imperfecti, molds, yeasts, stages, forms and spores should be considered as suitable for the practice of the present invention.

Suitable fungal species include by way of example only, but not of limitation: Agaricus augustus, A. blazei, A. brunnescens, A. campestris, A. lilaceps, A. placomyces, A. subrufescens and A. sylvicola, Acaulospora delicata; Agrocybe aegerita and A. arvalis; Albatrellus hirtus and A. syringae; Alpova pachyploeus; Amanita muscaria; Antrodia carbonica; Armillaria bulbosa, A. gallica, A. matsutake, A. mellea and A. ponderosa; Astraeus hygrometricus; Athelia neuhoffii; Auricularia auricula and A. polytricha; Bjerkandera adusta and B. adusta; Boletinellus merulioides; Boletus punctipes; Bondarzewia berkeleyi; Bridgeoporus nobilissimus; Calvatia gigantea; Cenococcum geophilum; Ceriporiapurpurea; Ceriporiopsis subvermispora; Collybia albuminosa and C. tuberosa; Coltricia perennis; Coniophoraputeana; Coprinus comatus and ‘Inky Caps’; Cordyceps variabilis, C. facis, C. subsessilis, C. myrmecophila, C. sphecocephala, C. entomorrhiza, C. gracilis, C. militaris, C. washingtonensis, C. melolanthae, C. ravenelii, C. unilateralis, C. clavulata and C. sinensis; Cyathus stercoreus; Daedalea quercina; Dentocorticium sulphurellum; Echinodontium tinctorium; Fistulina hepatica; Flammulina velutipes and F. populicola; Flavodonflavus; Fomes fomentarius; Fomitopsis officinalis and F. pinicola; Ganoderma applanatum, G. australe, G. curtisii, G. japonicum, G. lucidum, G. neo-japonicum, G. oregonense, G. sinense and G. tsugae; Gigaspora gigantia, G. gilmorei, G. heterogama, G. margarita; Gliocladium virens; Gloeophyllum saeparium; Glomus aggregatum, G. caledonius, G. clarus, G. fasciculatum, G. fasiculatus, G. lamellosum, G. macrocarpum and G. mosseae; Grifola frondosa; Hebeloma anthracophilum and H. crustuliniforme; Hericium abietes, H. coralloides, H. erinaceus and H. capnoides; Heterobasidion annosum; Hypholoma capnoides and H. sublateritium; Hypsizygus ulmarius and H. tessulatus (=H. marmoreus); Inonotus hispidus and I. obliquus; Irpex lacteus; Lactarius deliciosus; Laetiporus sulphureus (=Polyporus sulphureus); Lentinula edodes; Lentinus lepideus, L. giganteus, L. ponderosa, L. squarrosulus and L. tigrinus; Lentinula species; Lenzites betulina; Lepiota rachodes and L. procera; Lepista nuda (=Clitocybe nuda); Lycoperdon lilacinum and L. perlatum; Lyophyllum decastes; Macrocybe crassa; Marasmius oreades; Meripilus giganteus; Merulius tremellosus and M. incamatus; Morchella angusticeps, M. crassipes and M. esculenta; Mycena citricolor and M. chlorophos; Omphalotus olearius; Panellus stypticus; Paxillus involutus; Penicillium oxalicium; Phaeolus schweinitzii; Phellinus igniarius P. linteus and P. weirii; Pholiota nameko; Piloderma bicolor, Piptoporus betulinus; Pisolithus tinctorius; Pleurotus citrinopileatus (=P. comucopiae var. citrinopileatus), P. cystidiosus, (=P. abalonus, P. smithii (?)), P. djamor (=P. flabellatus, P. salmoneo-stramineus), P. dryinus, P. eryngii, P. euosmus, P. ostreatus, P. pulmonarius (=P. sajor-caju) and P. tuberregium; Pluteus cervinus; Polyporus indigenus, P. saporema, P. squamosus, P. tuberaster and P. umbellatus (=Grifola umbellata); Psathyrella hydrophila, Psilocybe aztecorum, P. azurescens, P. baeocystis, P. bohemica, P. caerulescens, P. cubensis, P. cyanescens, P. hoogshagenii, P. mexicana, P. pelliculosa, P. semilanceata, P. tampanensis and P. weilii; Rhizopogon nigrescens, R. roseolus and R. tenuis (=Glomus tenuis); Schizophyllum commune; Schizopora paradoxa; Sclerocytis sisuosa; Serpula lacrymans and S. himantioides; Scleroderma albidum, S. aurantium and S. polyrhizum; Scutellospora calospora; Sparassis crispa and S. herbstii; Stereum complicatum and S. ostrea; Stropharia aeruginosa, S. cyanea, S. albocyanea, S. caerulea and S. rugosoannulata; Suillus cothumatus; Talaromyces flavus; Termitomyces robustus; Trametes hirsuta, T. suaveolens and T. versicolor, Trichoderma viride, T. harmatum; Tricholoma giganteum and T. magnivelare (Matsutake); Tremella aurantia, T. fuciformis and T. mesenterica; Volvariella volvacea; and numerous other beneficial fungi.

For ecological restoration, all the fungi (including not only economically valuable species but also “little brown mushrooms” and “toadstools”) may play a valuable role, including stump and log dwelling fungi, wood chip dwelling fungi, ground dwelling fungi, mycorrhizal fungi and the fungi imperfecti. For example, spores or hyphae of the genus Morchella such as Morchella angusticeps, M. crassipes and M. esculenta, gourmet ground dwelling mushrooms that are known to favor fire-burned areas, may optionally be utilized in the present inventions in fire recovery efforts, thereby introducing a potential source of very rapidly growing mycelium into the soil at the same time seeds are introduced or landscaping cloths are laid. Preferred species for ecological restoration (and most other purposes) include Auricularia polytricha; Agaricus blazei and A. brunnescens; Agrocybe aegerita; Bridgeoporus nobilissimus; Coprinus comatus; Flammulina velutipes and F. populicola; Fomesfomentarius; Fomitopsis officinalis and F. pinicola; Ganoderma lucidum, G. oregonense and G. tsugae; Grifola frondosa; Hericium abietes and H. erinaceus, Hypholoma capnoides and H. sublateritium; Hypsizygus ulmarius and H. tessulatus; Laetiporus sulphureus; Lentinula edodes; Lepista nuda; Morchella angusticeps; Pholiota nameko; Pleurotus citrinopileatus, P. cystidiosus, P. eryngii, P. euosmus, P. ostreatus, P. pulmonarius and P. tuberregium; Polyporus umbellatus and P. tuberaster, Psilocybe azurescens, P. cubensis, P. cyanescens, P. mexicana, P. semilanceata and P. tampanensis (where these species are legal for such purposes); Sparassis crispa; Stropharia rugosoannulata; Trametes versicolor, Tremellafuciformis; and Volvariella volvacea.

Of particular use where insect pest control is desired are the entomopathogenic fungal species Metarhizium anisopliae, Metarhizium flaviride, Beauveria bassiana, Beauveria brongniartii, Beauveria amorpha, Pacilomyces fumosoroseus, Verticillium lecanii, Hirsutella citrifornis, Hirsutella thompsoni, Cordyceps variabilis, Cordycepsfacis, Cordyceps subsessilis, Cordyceps myrnecophila, Cordyceps sphecocephala, Cordyceps entomorrhiza, Cordyceps gracilis, Cordyceps militaris, Cordyceps washingtonensis, Cordyceps melolanthae, Cordyceps ravenelii, Cordyceps unilateralis and Cordyceps clavulata.

Preferred species for mycoremediation include the saprophytic mushrooms Fomes fomentarius (E. Coli and other bacteria, protists, pathogens etc.); Fomitopsis officinalis and F. pinicola; Ganoderma lucidum, G. oregonense and G. tsugae; Laetiporus sulphureus; Pleurotus ostreatus and the other Pleurotus species (oils, polyaromatic, alkane and alkene hydrocarbons including chlorinated compounds, brominated compounds, hormones, etc.); Polyporus umbellatus (malaria and other bacteria); Psilocybe azurescens and P. cyanescens (Sarin and VX and other phosphorylated nerve gases, organophosphate pesticides, etc.); Stropharia rugosoannulata (bacteria, urban and agricultural runoff, mycofiltration, as a “follow-up” species to Pleurotus and other white-rot fungi, etc.); and Trametes versicolor and other Trametes and species (Sarin, VX and other phosphorylated nerve gases, organophosphate pesticides, etc.), Collybia and the similar Marasmius and numerous “satellite genera” (metals, heavy metals, ores, etc.) as well as the other gilled and polypore genera and species listed above. Where the mycotechnologies of the present invention are utilized for remediation of toxic materials, the fungal species are preferably adapted to the substrate, that is cultured, fed (challenged with) the target contaminant(s) or substrates, selected for vigorous growth and thereby preconditioned to most effectively degrade the target substrates and/or contaminant(s). See Growing Gourmet and Medicinal Mushrooms, supra.

The species above include some of the many examples of the useful and beneficial fungi that may be utilized with the present invention; the scope of the invention as pertaining to fungi should not be considered thereby limited, as it will be recognized that all fungi may be favorably employed in the present invention.

By selecting the type of fungal spores or hyphae to be infused into the target, the course of colonization by fungi can be directed, allowing selection of economically or ecologically significant species of fungi, including mushrooms useful for ecological preservation, reforestation and habitat restoration, mushrooms useful for bioremediation of toxic wastes and pollutants, mushrooms with mycelia useful as an agricultural amendment, gourmet mushrooms, medicinal mushrooms containing valuable physiologically active compounds and pro-compounds, and mushrooms containing valuable enzymes, enzyme precursors and useful chemical compounds. Succession also occurs—as one type of mushroom exhausts its nutrient supply, another takes its place. To some degree, control of the successions of insect populations can also be achieved by selecting mosaics of fungal species which can predetermine species sequences. Fungal species may be selected for a specific environment, for example lawns, gardens, crop fields, forests (ranging from plains to mountainous to tropical ecosystems environments), aquatic environments including riparian, marsh, wetlands, estuaries, ponds, lakes, ditches, saline environments, etc.

A single species may be employed for a single application—for example, a single saprophytic species on a fiber substrate in conjunction with a single plant species such as Hypsizygus ulmarius on sawdust with corn. For typical ecological restoration, mycoremediation of toxic wastes, habitat restoration and preservation, etc., a plurality of species is preferred. The variety of species produce different species specific enzymatic systems that break down different chemicals and make these chemicals biologically available as nutrients for the microsphere and the biosphere. An example can be seen in the breakdown of a recalcitrant substrate—a hardwood such as ironwood, a substrate containing high concentrations of the complex polyaromatic cellulose carbohydrate compounds and the complex heterogeneous polyaromatic polymer lignin. A succession of mushrooms may be grown on the same wood, each species breaking down different compounds via different enzymatic systems, thereby making the carbon, nitrogen, phosphorus, hydrogen, etc. available as nutrients. To illustrate, a succession of gourmet mushroom species may be grown on the same wood. For example, Lentinula edodes (Shiitake) may be first grown on the wood, then Pleurotus ostreatus (Oyster), then Stropharia rugosoannulata (King Stropharia, Garden Giant or ‘Godzilla Mushrooms’), at which point the wood will have been transformed into a rich soil, suitable for gourmet mushrooms such as Coprinus comatus (Shaggy Mane). The same principle can be observed in nature where three or four different mushroom species may be observed fruiting from the same stump, each digesting a different woody compound and making the compounds available to the biosphere in the form of mycelium and mushrooms, or where different species of mushrooms may be observed fruiting from the floor of the forest adjacent to each other. The saprophytic mushrooms illustrated above also make such nutrients available to mycorrhizal fungi, thus further enhancing the symbiotic relationship with plants and resulting in greatly increased growth. Thus a plurality of fungal strains and species is often preferred, including, for example, the various saprophytic mushroom fungi and combinations of fungi including saprophytic-entomopathogenic, saprophytic-mycorrhizal, saprophytic-mycorrhizal-entomopathogenic, saprophytic-mycorrhizal-fungi imperfecti, etc., optionally packaged separately or in combination with seeds, the various fiber substrates, soils, etc.

It will be appreciated that many or all seeds or seedlings may be preferably employed with the present invention. While the totality of plants is too large to list, a few examples of native grass, sedge, rush and grass-like seeds and cultivated seeds include Agrostis exarata (Spike Bentgrass), Ammophila arenaria (European sand dune or beach grass), Ammophila breviligulata (American beach grass), Ammophila champlainensis Seymour, Ammophila maritima, Beckmannia zyzigachne (American Sloughgrass), Bromus carinatus (California Brome), Bromus vulgaris (Columbia Brome), Carex densa (Dense-Headed Sedge), Carexfeta (Green-Sheathed Sedge), Carex leporina (Harefoot Sedge), Carex lenticularis (=C. kelloggiz) (Shore Sedge), Carex lyngbyel (Lyngby Sedge), Carex macrocephala (Big Headed Sedge), Carex obnupta (Slough Sedge), Carex pansa (Foredune Sedge), Carex unilateralis (One-Sided Sedge), Deschampsia caespitosa (Tufted Hair Grass), Eleocharis palustis (Creeping Spike rush), Elymus glaucus (Blue Wild Rye), Festuca idahoensis-var. roemeri (Roemer's Fescue), Festuca rubra var. littoralis (Shore Fescue), Festuca subulata (Bearded Fescue), Glyceria elata (Tall Mannagrass), Glyceriaoccidentalis (Western Mannagrass), Hordeum brachyantherum (Meadow Barley), Juncus effusus (Soft Rush), Juncus patens (Spreading Rush), Juncus tenuis (Slender Rush), Lozula campestris (Woodrush), Phalaris arundinacea (Reed Canary Grass), Phalaris aquatica, Phalaris tuberosa (Staggers Grass), Phalaris canariensis, Poa Macrantha (Dune Bluegrass), ReGreen (Sterile Hybrid Wheat), Scirpus acutus (Hardstem Bullrush), Scirpus americanus, Scirpus cyperinus, Scirpus maritimus (Seacoast Bullrush), Scirpus microcarpus, Scirpus validus, Sparaganuim eurycarpum (Giant Burreed), Triglochin maritinum (Seaside Arrowgrass), Typha latifolia (Cattail), Alopecuris geniculatus, Carexpachystachya, Carex stipata (grass like), Danthonia califomica, Eleocharis ovata (grass like), Glycaria grandis, Juncus acuminatus, Juncus bolanderi and Juncus ensifolius (Daggar leaf rush).

Example applications include: 1) Habitat recovery/reclamation: ‘regreening’ of roads, especially logging roads, important in lands returned to wilderness or wildlife preserves and for prevention of sediment and silt runoff into waterways from existing gravel roads, depleted environments, scarred or biologically hostile environments, all typically lacking topsoils. For example, a preferred method of restoration on top of gravel logging roads would be to lay down a 2.5-10 cm. (1-4 inch) layer of mixed wood chips (i.e. hog fuel type wood chips), broadcast saprophytic and mycorrhizal species either by free hand, hydroseeding or via mycocloths or mycobags (or any combination thereof or via other mycotechnologies discussed herein), grass seeds are applied, and then chopped straw, twigs, etc. loosely overlaid over the top surface to provide shade and moist air pockets. If a non-seeding, non-native grass, is used the first year, the carbon cycle is begun, and as they mature, decline and die, the newly available debris further fuels the carbon cycle. By using a light infusion of native seeds and/or seeds or seedlings of shrubs and trees, or by depending upon natural re-seeding from adjacent lands, this method will stimulate the process of habitat restoration leading to a more native environment. The process of soil generation is sped up by months, releasing nutrients to benefit plants and other organisms. This process creates topsoils and encourages biological recovery and complexity. The mycelium retains sediments and silts washed from the gravel road, incorporating them into topsoil while preventing release into waterways. This is also useful as a method of accumulating carbon credits.; 2) Mycofiltration: protection of sensitive watersheds and ecosystems from upland or neighboring sources/vectors of contamination by capturing in the mycelial network. This is critical for urban developments, protection of salmon or trout streams, estuary environments, etc.; 3) Mycobags, mycogabions, mycocloths and mycobags overlaying toxic waste fields: penetration of mycelium to several inches is achieved, a year later, decontaminated soil can be scooped up (now a value added product), and then another layer of mycobags, mycogabions, etc. can be placed on top. This can be done sequentially for the deep removal of toxins.; 4) Saprophytic, mycorrhizal-saprophytic-entomopathogenic, saprophytic-entomopathogenic and other fungally inoculated substrates for environmental and agricultural enhancement and control of pest microorganisms and insects; 5) Soil regeneration and reforestation via burlap bags inoculated with fungi and layered over the ground with hybrid poplars planted 6-12 feet apart; 6) Deep trenching wherein a narrow, deep ravine is filled with sawdust, woodchips, straw and/or agricultural wastes and inoculated with mycelium; 7) Chicken (and other animal) farms where waste exceeds the capacity to recycle, resulting in phosphorus and nitrogen devastating the watershed. Mycofiltration is achieved via creation of ‘mycological parks’ utilizing species suited to the local environmental conditions and wastes/nutrient materials for fungal growth). For example, in the southeastern United States, Pleurotus ostreatus and P. eryngii, Coprinus comatus and Agaricus brunnescens, A. blazei and A. bitorquis could be used for sheet inoculation, covered with 5-15 cm. (2-6 inches) of chicken/sawdust waste. Poplars, cottonwoods and other trees could be planted for hydraulic control and protection of groundwater; 8) A cardboard insect monitoring station utilizing mycoattractants such as extracts of pre-conidial mycelia and/or pre-conidial mycelia of mycopesticidal, entomopathogenic fungi such as Metarhizium anisopliae, Beauveria bassiana, Paecilomyces and Cordyceps species. Since the targeted insects respond to and are drawn towards the loci of the extracts, the extracts can be presented in a wide variety of ways and still demonstrate attractancy. The insect myco-attractant may be saturated into a wicking agent or membrane to slowly out-gas the attractant fragrance. The surface area of the membrane or wick, its absorptive properties, its rate of release of volatile attractants and the duration of wicking are all influenced and easily altered according to the target insect and environmental considerations. The monitoring station would then register ‘hits’ by registering by any means the numbers of visitations from the insects. This sampling can be indispensable for recommending subsequent treatments; 9) Empowering other insect treatment and control systems. The soaking of mycoattractant extract onto cellulose, paper, cardboard, wood or other biodegradable materials for a period of time and at a concentration to be effective allows for construction of a biodegradable monitoring or kill station. The insects, such as termites, fire ants and carpenters ants, enter into a chamber where the mycoattractant is localized and then are trapped and/or killed via ingestion of the material containing mycopesticidal extract. Alternatively, the target insects are attracted to the monitoring station, trap or to a close proximity where they are captured and/or killed via any insect treatment or control means, including but not limited to the use of adhesives, electricity, moving air, sprays, chemicals (toxins, growth regulators, for instance), desiccants, cold temperatures, hot air, mechanical devices and combinations thereof. Such monitors or traps can be useful to analyzing, treating and solving the problems associated with invasive insects, and is highly applicable to rural, agricultural, forested, urban and suburban settings. 10) Controlling social insects such as fire ants, carpenter ants and termites with the construction of monitoring and/or killing stations utilizing extracts of the pre-conidial mycelia of mycopesticidal, entomopathogenic fungi combined with pre-conidial mycelium of such fungi on a biodegradable cellulosic material like wood, paper or cardboard. This combination of extract and live mycelium has two advantages. The target insects are attracted to the locus from which the fragrance of the extract emanates. As the mycelia grows, it also outgases an attractant fragrance. The insect consumes the extract-impregnated cellulose and also makes contact with fragments of mycelia. As the insect travels, mycelia is spread. As the insect weakens with illness, the mycelia becomes stronger. The insect is killed by both exposure to the attractant but toxic extract and from infectious colonization by the fungus. The time delay of exposure to death is an added advantage as it allows the infected individuals to fully disperse through the affected region as well as the nest without being sequestered and expunged from the colony; 11) The use of mycoattractants derived from the extracts of the mycelia of pre-conidial, entomopathogenic, mycopesticidal fungi to place ‘bait stations’ having these extracts in strategic locations to draw in insect plagues to a single locus. Locust plagues could be diverted and drawn towards 55 gallon drums hosting the mycoattractants wherein the insects could be trapped. Mycelially based extracts of pre-conidial mycelium of entomopathogenic fungi could be utilized to prevent plagues, herd insects to control points, avoiding massive crop damage and economic devastation, and negating the need for costly and toxic chemicals; 12) The use of mycoattractants derived from the extracts of the mycelia of pre-conidial, entomopathogenic, mycopesticidal fungi to draw in beneficial insects whose predatory preferences include the plague insect. For instance, a gardener could increase the number of lady bugs if aphid infestations get out of control; and 13) The use of attractant emitters using extracts of pre-conidial mycelium from mycopesticidal, entomopathogenic fungi to attract pollinating insects to disadvantaged plants by placing them in close proximity of the targeted plants. This invention will be become increasingly important with the loss of sufficient populations of insects which would otherwise naturally accomplish the task of pollination.

### EXAMPLE 1

A coconut fiber door mat was pressure steam-sterilized in a polypropylene bag at 1 kg/cm² (15 psi) for two hours, inoculated with rye grain spawn, and the fungus allowed to overgrow the mat. Grass seeds were added and the mat moved to an outdoor location. The mat was observed to fruit Pleurotus ostreatus (Oyster) mushrooms and the seed was observed to sprout and prosper. Birds were observed hunting for grass seed in the mycomat; they appeared to prefer feeding from the fungal mat as compared to feeding from a nearby (15 feet) bird feeder. The birds were observed to add bird guano to the mat, thereby increasing the nutritional base and introducing various organisms to the biological community.

### EXAMPLE 2

Grain spawn of Pleurotus ostreatus was layered between straw-coconut fiber mats steam-sterilized as above. Oyster mushrooms pushed through the un-colonized upper layer of the straw-coconut fiber mat, resulting in ‘island fruitings’ scattered over the mats with a heavy dusting of spores dispersed around the mushrooms. These parents provided the means for subsequent and more thorough colonization. This sandwich inoculation provides an extremely efficient use of spawn, with sheet inoculation of thin layer(s) of spawn producing a prodigious amount of spores and numerous satellite colonies of inoculated substrate.

### EXAMPLE 3

By introducing spores of Stropharia rugosoannulata, an edible mushroom, into hydroseeding mulch materials, the receiving fabric material, straw and wood chips soon colonized with mycelium. Plant growth was enhanced, as well as water retention, and eventually edible mushrooms were produced. Bees were attracted to the mycelium and fly larvae hatched from the mushrooms along the stream bank, the larvae and resultant insects providing a benefit to fish. In two years the wood chips had become rich soil.

The present invention utilizes the design and active insertion of individual saprophytic, mycorrhizal, entomopathogenic, and parasitic fungal species and mosaics of species to catalyze habitat recoveries from catastrophia. Furthermore, by using delivery systems and mycotechnologies disclosed herein instead of relying on serendipitous sporefalls, environmental designers can greatly benefit by establishing, strengthening or steering the course of habitat evolution in a fashion that is both environmentally sound and/or economically profitable. In installing new parks, landscapes, forests, arboretums, habitat oases and oasis-islands, space colonies, terrestrial environments on this planet and on others, the insertion of purposely designed ‘fungal footprints’ can dramatically improve the biodynamics of any ecosystem.

It should be understood the foregoing detailed description is for purposes of illustration rather than limitation of the scope of protection accorded this invention, and therefore the description should be considered illustrative, not exhaustive. The scope of protection is to be measured as broadly as the invention permits. While the invention has been described in connection with preferred embodiments, it will be understood that there is no intention to limit the invention to those embodiments. On the contrary, it will be appreciated that those skilled in the art, upon attaining an understanding of the invention, may readily conceive of alterations to, modifications of, and equivalents to the preferred embodiments without departing from the principles of the invention, and it is intended to cover all these alternatives, modifications and equivalents. Accordingly, the scope of the present invention should be assessed as that of the appended claims and any equivalents falling within the true spirit and scope of the invention.

I claim:

1. A method for filtering and controlling silts and sediments in water runoff utilizing saprophytic fungi, the method comprising:
a) inoculating a substrate with a saprophytic mushroom species to form an inoculated spawn; and
b) utilizing the inoculated spawn to inoculate a lignin- and cellulose-containing substrate applied to an area exposed to water runoff containing sediments and silts.

2. The method for filtering and controlling silts and sediments of claim 1 wherein the lignin- and cellulose-containing substrate is selected from the group consisting of wood and straw.

3. The method for filtering and controlling silts and sediments of claim 1 wherein the lignin- and cellulose-containing substrate is applied to an area exposed to water runoff selected from the group consisting of logging roads and gravel roads.

4. The method for filtering and controlling silts and sediments of claim 1 wherein the water runoff is into a habitat selected from the group consisting of watersheds, streams, salmon and trout spawning grounds, riparian runoff and wetlands.

5. The method for filtering and controlling silts and sediments of claim 1 wherein the method additionally comprises adding plant sources selected from the group consisting of seeds and seedlings and combinations thereof.

6. The method for filtering and controlling silts and sediments of claim 1 wherein the seeds and seedlings are selected from the group of plants consisting of garden vegetables, agricultural crops, grasses, herbs, shrubs, and trees.

7. The method for filtering and controlling silts and sediments of claim 1 wherein the saprophytic fungi is selected from the group consisting of:
a) gilled mushrooms including Agaricus, Agrocybe, Armillaria, Clitocybe, Collybia, Conocybe, Coprinus, Flammulina, Giganopanus, Gymnopilus, Hypholoma, Inocybe, Hypsizygus, Lentinula, Lentinus, Lenzites, Lepiota, Lepista, Lyophyllum, Macrocybe, Marasmius, Mycena, Omphalotus, Panaeolus, Panellus, Pholiota, Pleurotus, Pluteus, Psathyrella, Psilocybe, Schizophyllum, Sparassis, Stropharia, Termitomyces, Tricholoma, Volvariella and combinations thereof;
b) polypore mushrooms including Albatrellus, Antrodia, Bjerkandera, Bondarzewia, Bridgeoporus, Ceriporia, Coltricia, Daedalea, Dentocorticium, Echinodontium, Fistulina, Flavodon, Fomes, Fomitopsis, Ganoderna, Gloeophyllum, Grifola, Hericium, Heterobasidion, Inonotus, Irpex, Laetiporus, Meripilus, Oligoporus, Oxyporus, Phaeolus, Phellinus, Piptoporus, Polyporus, Schizopora, Trametes, Wolfiporia and combinations thereof;
c) Basidiomycetes including Auricularia, Calvatia, Ceriporiopsis, Coniophora, Cyathus, Lycoperdon, Merulius, Phlebia, Serpula, Sparassis and Stereum;
d) Ascomycetes including Cordyceps, Morchella, Tuber, Peziza and combinations thereof; and
e) jelly fungi including Tremella.

8. The method for filtering and controlling silts and sediments of claim 1 wherein the saprophytic fungi comprises a mushroom species selected from the group consisting of Pleurotus species, Trametes species, Ganoderma species, Collybia species, Fomes fomentarius, Fomitopsis officinalis, Fomitopsis pinicola, Stropharia rugosoannulata, Phellinus igniarius, Phellinus linteus, Psilocybe azurescens, Psilocybe cyanescens and Coprinus comatus.

9. The method for filtering and controlling silts and sediments of claim 1 wherein the lignin- and cellulose-containing substrate is also inoculated with a fungus selected from the group consisting of mycorrhizal fungi, entomopathogenic fungi, parasitic fungi, fungi imperfecti and combinations thereof.

10. The method for filtering and controlling silts and sediments of claim 9
a) wherein the mycorrhizal fungi is selected from the group consisting of the mycorrhizal mushrooms and endomycorrhizal and ectomycorrhizal non-mushroom fungi including Acaulospora, Alpova, Amanita, Astraeus, Athelia, Boletinellus, Boletus, Cantharellus, Cenococcum, Dentinum, Gigaspora, Glomus, Gomphidius, Hebeloma, Lactarius, Paxillus, Piloderma, Pisolithus, Rhizophagus, Rhizopogon, Rozites, Russula, Sclerocytis, Scleroderna, Scutellospora, Suillus, Tuber and combinations thereof;
b) wherein the entomopathogenic fungi is selected from the group consisting of entomopathogenic fungi including Metarhizium, Beauveria, Paecilomyces, Verticillium, Hirsutella, Aspergillus, Akanthomyces, Desmidiospora, Hymenostilbe, Mariannaea, Nomuraea, Paraisaria, Tolypocladium, Spicaria, Botrytis, Rhizopus, the Entomophthoracae and other Phycomycetes, Cordyceps and combinations thereof; and
c) wherein the fungi imperfecti is selected from the group consisting of the fungi imperfecti and related molds and yeasts including Actinomyces, Alternaria, Aspergillus, Botrytis, Candida, Chaetomium, Chrysosporium, Cladosporium, Cryptococccus, Dactylium, Doratomyces (Stysanus), Epicoccum, Fusarium, Geotrichum, Gliocladium, Humicola, Monilia, Mucor, Mycelia Sterilia, Mycogone, Neurospora, Papulospora, Penicillium, Rhizopus, Scopulariopsis, Sepedonium, Streptomyces, Talaromyces, Torula, Trichoderma, Trichothecium, Verticillium and combinations thereof.

11. A method for protecting habitats by filtering and controlling silts and sediments, the method comprising applying at least one layer of wood or straw substrate to a surface exposed to water runoff containing silts and sediments, inoculating the wood or straw substrate with a saprophytic mushroom species and a mycorrhizal fungi, and adding plant sources selected from the group consisting of seeds, seedlings, garden vegetables, agricultural crops, grasses, herbs, shrubs, trees and combinations thereof.
